(19) United States
(12) Patent Application Publication
(10) Pub. No.: US 2024/0066260 A1
(43) Pub. Date: Feb. 29, 2024

(54) PROVISION OF SESSIONS WITH INDIVIDUALLY TARGETED VISUAL STIMULI TOALLEVIATE CHRONIC PAIN IN USERS

(71) Applicant: Click Therapeutics, Inc., New York, NY (US)

(72) Inventors: Jacqueline LUTZ, Cambridge, MA (US); Samantha ADLER, Austin, TX (US)

(73) Assignee: Click Therapeutics, Inc., New York, NY (US)

(21) Appl. No.: 18/237,565

(22) Filed: Aug. 24, 2023

Related U.S. Application Data
(60) Provisional application No. 63/400,927, filed on Aug. 25, 2022.
(60) Provisional application No. 63/452,359, filed on Mar. 15, 2023.

(51) Int. Cl. A61M 21/00; A61B 5/00; A61B 5/16; G06F 3/01; G16H 20/70

(52) U.S. Cl. CPC A61M 21/00; A61B 5/163; A61B 5/4836; G06F 3/013; G16H 20/70; G16H 20/10

(57) ABSTRACT

Presented herein are systems and methods of providing individualized sessions to address chronic pain in users. A computing system may identify a set of stimuli identified by a user as not associated with the pain or the condition of the user. The computing system may present a first visual stimulus associated with the chronic pain of the user and a second visual stimulus neutral to the chronic pain. The computing system may present a visual probe corresponding to one of the first position or the second position to direct the user to interact with the visual probe. The computing system may increase the efficacy of the medication that the user is taking to address the condition.

### CROSS REFERENCE TO RELATED APPLICATIONS

The present application claims priority to U.S. Provisional Patent App. No. 63/400,927, filed Aug. 25, 2023, and to U.S. Provisional Patent App. No. 63/452,359, filed Mar. 15, 2023, each of which is incorporated herein by reference in their entireties.

### BACKGROUND

Certain conditions may cause a patient to direct attention to certain biases which may ultimately exacerbate their symptoms. Patients may simultaneously suffer from chronic pain, fear, and mood symptoms, such as in the case of patients experiencing Rheumatoid Arthritis, Diabetic Neuropathy, Fibromyalgia, or Irritable Bowel Syndrome (IBS), among other conditions. The desire to give attention to pain, fear, or other biases is innate, and further, patients can become hyper-aware of biases corresponding to related stimuli. For example, a patient experiencing Fibromyalgia may pay more attention to pain-related stimuli than a patient without chronic pain. In a similar manner, hypersensitivity to negative or pain-related stimuli can exacerbate fear in a patient. For example, a patient experiencing hypersensitivity may seek out signs of the pain, unlike a patient without such conditions. This attention to negative stimuli can cause worsened symptoms for the patient. For example, a bias towards pain can lead towards hypersensitization for the patient. Furthermore, these hypersensitivities can worsen the condition through fear-avoidance. In fear-avoidance, individuals can avoid stimuli or activities which are perceived to potentially cause pain, further weakening physical or mental conditions from the lack of activity.

Treating attentional biases in patients with chronic conditions can prove difficult due to the reinforcement of the association between the pain and the user formed in the mind of the user, often caused by these conditions. Although there are in-person behavioral therapies, frequent and immediate access to these behavioral therapies can be difficult to obtain, especially for a patient experiencing a chronic pain related condition such as Rheumatoid Arthritis, Diabetic Neuropathy, Fibromyalgia, or IBS, among others. The therapies may also not prove useful for the individual without consistent adherence, which may be difficult to guarantee due to the nature of pain and fear. Lastly, it can be difficult to ascertain which therapies would be the most beneficial to an individual, or if a combination of therapies would be the most beneficial.

Furthermore, stimuli provided via tasks to address the attentional biases related to the chronic pain may be ineffective at actually priming or training the patient to turn attention away from the stimuli associated with the chronic pain. The stimuli may be ineffective, because these may not be particularly targeted or personalized at the association formed in the mind of the patient between the patient's chronic pain and the stimulus. For example, a patient can have an association between the patient's own pain and a word such as “sharp,” but lack any association with words related to a different type of pain such as “cramping” that the patient is not experiencing. As a result, providing stimuli unrelated to the patient's individualized pain may be ineffective at addressing the attentional bias on the part of the user as well as a waste of time and resources on the part of the provider of the stimuli.

In addition, addressing such attentional biases related to chronic conditions in patients digitally through a device to present these therapeutic exercises can present a multitude of problems. For one, the user may be unable or have extreme difficulty refraining from paying mind to negative stimuli, thereby ignoring any attempts at intervention from a clinician or through a device. The user may thus find it difficult to adhere to treatments through digital means due to the nature of the chronic condition, leading to ineffective clinical outcomes. For another, it may be actually difficult for the patient to contact a clinician to receive treatment.

### SUMMARY

To resolve these and other technical challenges, a digital therapeutic treatment can be provided using visual stimuli targeted at the user's individualized association with the chronic pain to implement an attention-bias modification training (ABMT). Prior to performing the ABMT tasks, the user may be prompted to indicate (e.g., via interaction with a display or eye gaze) a degree of personal association between a visual stimulus (e.g., words or images) with the user's individualized chronic-pain related condition. Stimuli that are potentially related to but indicated as not associated with the pain by the user may be excluded or filtered out from provision. After this filtering process, the user may be repeatedly provided with curated ABMT sessions with individually-targeted visual stimuli, personalized for the user based on the user's own associations, as well as user's condition, user performance in prior sessions, and user input, among others, as part of the digital therapeutic. The repeated customized ABMT sessions can train the user to re-orient attention away from negative stimuli and instead turn the user's attention towards positive or neutral stimuli with respect to the chronic-pain related condition. In this manner, the user can be conditioned to pay less attention to the negative stimuli, such as pain- or stress-related stimuli, at a speed, frequency, and efficacy available through the digital therapeutic application. Additionally, by excluding visual stimuli identified as not associated with the individualized pain of the user, the probability of sending ineffective stimuli can be reduced, thereby lowering unnecessary consumption of computing resources of the user device providing the stimuli.

Through a customized attention-bias modification training (ABMT) approach including individually-targeted visual stimuli, the user's ability to redirect attention from negative stimuli may be increased. Controlling the bias towards negative stimuli can be a facet of remediating or resolving symptoms of a condition such as Fibromyalgia, IBS, Rheumatoid Arthritis, or diabetic neuropathy, or other conditions associated with chronic pain. The ABMT can be a type of behavioral therapy in which a patient is trained to decrease attention or thought paid to negative aspects of their condition, such as pain, through performance of tasks. By performing tasks related to the condition, the user's neural system may be primed or trained to reduce bias, or propensity, towards negative associations, thereby enabling the user to focus less on the condition and its symptoms and reduce recurrent thoughts of the condition when presented with stimuli related to the condition. In this manner, the user may reduce overall symptomology of the condition, such as pain, by training the user to more easily refocus on neutral or positive stimuli over negative stimuli associated with the condition.

An example of ABMT can apply dot probe tasks in order to train the user away from the negative attention bias associated with the user's condition. A dot probe task can include a visual presentation of a set point or fixation point on a screen. Other visual stimuli can present themselves in relation to the fixation point, which remains at the same location on the screen, regardless of the other visual stimuli. During a dot probe task, the user may focus on the fixation point on the screen. The digital therapeutics application may present visual stimuli in conjunction with the fixation point. As a therapeutic approach, two stimuli presented as images or words can be presented to the user. The first stimulus can be a negative stimulus, or a stimulus associated with the condition, as previously indicated by the user. For example, the first stimulus can include the words “pain,” “disease,” or “tired.” Stimuli while potentially related to but indicated as not associated with the pain by the user may be excluded or filtered out from provision. This can allow for selection of visual stimuli targeted at the user's own associations with the pain and prevent formation of new associations in their mind with respect to the pain. The second stimulus can be a positive or neutral stimulus unassociated with the condition. For example, the second stimulus can include the words “love,” “good,” or “happy.” The two stimuli can be presented to the subject in addition to the fixation point.

During the dot probe task, the visual stimuli may be presented for a period of time before disappearing from the screen. The user may then be prompted through the application to interact with the device. For example, upon the removal of the visual stimuli from presentation on the screen, the digital therapeutics application may present a visual probe in relation to the fixation point. The visual probe may be presented at a location associated with the prior presentation of visual stimuli. For example, the visual probe may be presented in a location where a positive or neutral stimuli had been prior presented. The user may be prompted to interact with the application upon the presentation of the visual probe, such as by selecting the visual probe or otherwise interacting with the application. By interacting with visual probes which are associated with positive or neutral stimuli, the user can be trained to see more clearly, pay more attention to, and otherwise be more inclined to notice the positive or neutral stimulus over the negative stimulus through repeated tasks.

In addition, presentation of tasks of an ABMT approach can be tailored based on the user's responses. The system can alter characteristics of the visual stimuli, including the placement, color, size, font type, image, or duration of presentation, to best train the user away from negative biases associated with his condition. Each interaction (or non-interaction in some cases) with the digital therapeutics application can cause a response by which the system can determine parameters for presentation of the tasks to the user. Time between the presentation of the visual stimuli, presentation of the visual prompt, or receipt of the interaction, among others, can be used to determine subsequent tasks. Furthermore, a metric can be determined for the response based on time between presentations of visual stimuli or prompts and the interaction.

By providing personalized visual stimuli during an ABMT, the user's ability to resist a bias towards negative stimuli may be increased through modification of biases. Resisting the bias towards negative stimuli can be a facet of remediating or resolving symptoms of a chronic condition such as Rheumatoid Arthritis, Diabetic Neuropathy, Fibromyalgia, or IBS, as examples. As the user progresses through tasks of the session, the tasks may increase in difficulty. An increase in difficulty can be associated with less display time of the visual stimuli, less display time of the visual prompt, or more closely related or similar visual stimuli. More closely related or similar visual stimuli can refer to text which resembles other text more closely, such as in length, number of characters, pronunciation, similarity in definition, or similarity or repetition of characters, among others. By presenting two or more visual stimuli for a period of time, the user can perform a task such as interacting with a visual probe associated with the positive or neutral stimulus to turn the user's attention towards the neutral or positive stimulus. Upon choosing the correct (e.g., positive or neutral) visual probe, the user can receive positive feedback to bias the user towards a positive or neutral stimulus and away from the negative stimulus. Through this method, the user can be trained to focus on the image associated with the positive or neutral stimulus. The personalization of the visual stimuli as part of the digital therapeutic can greatly reduce the bias towards negative stimuli or reduce the bias away from positive stimuli.

To provide this therapy, the computing system may select two or more visual stimuli including at least two words or images and associated actions for the user to transmit to the end user device. The computing system may have filtered out or excluded other negative stimuli that were indicated by the user as not associated with their condition or chronic pain experience. The computing system may have received preferences from the user, such as a preferred list of words or images, or a rating of the association of the presented stimuli with a negative or positive connotation for the user. From the remaining set, the computing system may select a stimulus negatively associated with the user's condition, as indicated by the user as associated with the condition. In addition, the computing system may select a positive or neutral stimulus to be presented with the negative stimulus. Furthermore, as the computing system acquires additional data about the user, the computing system may be able to select stimuli more targeted toward the specific user and their condition and may store this data in a profile of the user. The computing system may select a subsequent stimulus based on at least the prior stimuli, the completion of the prior action, the profile of the user, or an evaluation of the user's performance with prior stimuli, among others.

In this manner, the user can be provided with targeted stimuli relating to the chronic condition with ease to help retrain a bias towards negative stimuli relating to the condition as documented herein. By selecting the stimuli sent to the user to address the subject's bias towards negative stimuli, the quality of human computer interactions (HCI) between the user and the device may be improved. In addition, since the stimuli are more related to the user's condition, unnecessary consumption of computational resources (e.g., processing and memory) of the computing system and the user device and the network bandwidth may be reduced, relative to sending ineffective messages.

Furthermore, in the context of a digital therapeutics application, the individualized selection of targeted visual stimuli as part of the ABMT can be directed at the user's particular association between visual stimuli and the user's chronic pain. This individualization may result in the delivery of user-specific interventions to improve subject's adherence to the digital therapeutic treatment. The improved adherence may result in not only higher adherence to the therapeutic interventions but also potential improvements to the subject's bias towards negative stimuli. Also, since the digital therapeutics application operates on the subject's device, or at least a device that the user can access easily and reliably (e.g., according to the predetermined frequency such as once per day), the application can provide real-time support to the subject. For example, upon receiving a request from the user to initiate a session, the application initiates a session in near-real time. Such prompt guidance cannot be achieved via in-person visits, phone calls, video conferences or even text messages between the user and health care providers examining the user for the underlying condition. Due to this accessibility, the application is able to provide and customize tasks for the user based on the performance of the user. This can create an iteratively improving service for the user wherein overall bandwidth and data communications are minimized due to the increasing usefulness of each session.

Aspects of the present disclosure relate to systems and methods for providing sessions to address chronic pain in users. The system may include a computing system having one or more processors coupled with memory. The computing system may identify, for a session to address chronic pain in a user, (i) a first visual stimulus associated with the chronic pain and (ii) a second visual stimulus being neutral with respect to the chronic pain. The computing system may present, relative to a fixation point on a display, the first visual stimulus at a first position and the second visual stimulus at a second position during the first portion of the session. The computing system may remove, from presentation on the display, the first visual stimulus and the second visual stimulus subsequent to elapsing of the first portion. The computing system may present a visual probe corresponding to one of the first position or the second position relative to the fixation point, to direct the user to interact with the visual probe during a second portion of the session. The computing system may determine a response by the user to presentation of the visual probe. The computing system may provide a feedback indication for the user based on the response by the user.

In some embodiments, the computing system may identify, for each visual stimulus of a plurality of visual stimuli, an indication of a value identifying a degree of association of the corresponding visual stimulus with the chronic pain for the user based on at least one of (i) an interaction with a user interface or (ii) an eye gaze with respect to the corresponding visual stimulus displayed on the user interface. The computing system may select the first visual stimulus from the plurality of visual stimuli based on a corresponding value for the visual stimulus satisfying a threshold. In some embodiments, the computing system may exclude, from a set of visual stimuli, at least one visual stimulus for presentation to the user, responsive to a corresponding value of the at least one visual stimulus not satisfying the threshold.

In some embodiments, the computing system may determine that the response by the user is correct, responsive to the user interacting with the visual probe where the second visual stimulus being neutral with respect to the chronic pain was presented on the display. The computing system can generate the feedback indication based on the determination that the response is correct. In some embodiments, the computing system may determine that the response by the user is incorrect, responsive to the user interacting on the display outside a threshold distance away from where the second visual stimulus being neutral with respect to the chronic pain was presented on the display. The computing system can generate the feedback indication based on the determination that the response is incorrect.

In some embodiments, the computing system may select a visual characteristic for the visual probe based on a visual characteristic of the fixation point presented on the display. In some embodiments, the computing system may determine to provide the session to the user in accordance with a session schedule. The session schedule may identify a frequency over a time period at which the user is to be provided with the session. In some embodiments, the computing system can identify the first visual stimulus and the second visual stimulus by selecting, from a set of stimulus types, a first stimulus type for the session based on a second stimulus type selected for a prior session. In some embodiments, the set of stimulus types may include a text stimulus type, a scenic image stimulus type, a facial expression image stimulus type, or a video stimulus type.

In some embodiments, the computing system may identify an eye gaze of the user as toward one of the first visual stimulus associated with the chronic pain or the second visual stimulus being neutral with respect to the chronic pain. In some embodiments, the computing system may determine that the response is correct, responsive to identifying an eye gaze of the user as towards the second visual stimulus being neutral with respect to the chronic pain. Providing the feedback indication for the user may include the computing system generating the feedback indication based on the determination that the response is correct. In some embodiments, the computing system may determine that the response is incorrect, responsive to identifying an eye gaze of the user as towards the first visual stimulus being associated with the chronic pain. Providing the feedback indication for the user may include the computing system generating the feedback indication based on the determination that the response is incorrect.

In some embodiments, the computing system may modify a session schedule identifying a frequency over a time period at which the user is to be provided with the session based on a rate of correct responses by the user. In some embodiments, the computing system can provide the feedback indication based on a time elapsed between the presentation and the interaction. In some embodiments, the user may be on a medication to address the chronic pain associated with a condition, at least in partial concurrence with the session. The chronic pain associated with the condition may cause the user to have attention bias towards stimuli associated with the chronic pain. The condition may include at least one of rheumatoid arthritis, irritable bowel syndrome, fibromyalgia, or diabetic neuropathy.

Aspects of the present disclosure relate to systems and methods for alleviating chronic pain associated with a condition in a user in need thereof. A computing system may obtain a first metric associated with the user prior to a set of sessions. The computing system may repeat, for each session of the set of sessions, (i) presentation, during a first portion of the session via a display, a respective set of visual stimuli comprising (a) a first visual stimulus associated with the chronic pain at a first position and (b) a second visual stimulus that is neutral with respect to the chronic pain at a second position, relative to a fixation point presented on the display; (ii) removal, from presentation on the display, the first visual stimulus and the second visual stimulus subsequent to the elapsing of the first portion; and (iii) presentation, during a second portion of the session via the display, a visual probe corresponding to one of the first position or the second position relative to the fixation point, to direct the user to interact with the visual probe. The computing system may obtain a second metric associated with the user subsequent to at least one of the set of sessions. The chronic pain associated with the condition is alleviated in the user when the second metric is (i) decreased from the first metric by a first predetermined margin or (ii) increased from the first metric by a second predetermined margin.

In some embodiments, the condition may include at least one of rheumatoid arthritis, irritable bowel syndrome, fibromyalgia, or diabetic neuropathy. In some embodiments, the chronic pain associated with the condition may cause the user to have attention bias towards stimuli associated with the chronic pain. In some embodiments, the user may be on a medication to address the chronic pain associated with the condition, at least in partial concurrence with at least one of the set of sessions. In some embodiments, the medication can include at least one of acetaminophen, a non-steroidal anti-inflammatory drug (NSAID), or an anticonvulsant.

In some embodiments, the chronic pain can be alleviated in the user, when the second metric is increased from the first metric by the second predetermined margin. The first metric and the second metric can be pain self-efficacy values. In some embodiments, the condition in which chronic pain is alleviated based on the pain self-efficacy values can include rheumatoid arthritis. In some embodiments, the chronic pain can be alleviated in the user, when the second metric is decreased from the first metric by the first predetermined margin. The first metric and the second metric can be pain catastrophizing scale values.

In some embodiments, the pain catastrophizing scale values for the first metric and the second metric may include at least one of a value for helplessness, a value for rumination, or a composite value. In some embodiments, the condition in which chronic pain can be alleviated based on the pain catastrophizing scale values for rumination can include fibromyalgia. In some embodiments, chronic pain associated with rheumatoid arthritis can be alleviated in the user, when the second metric is decreased from the first metric by the first predetermined margin. The first metric and the second metric can be brief pain inventory interference (BPI-I) values.

In some embodiments, chronic pain associated with rheumatoid arthritis can be alleviated in the user, when the second metric is increased from the first metric by the second predetermined margin. The first metric and the second metric can be brief patient-reported outcomes measurement information system (PROMIS) values for social participation. In some embodiments, the set of sessions may be provided over a period of time ranging between 1 to 90 days, in accordance with a session schedule. In some embodiments, the first visual stimulus and the second visual stimulus in the respective set of stimuli in each session may be both of a stimulus type of a set of stimulus types. The set of stimulus types may include a text stimulus type, a scenic image stimulus type, a facial expression image stimulus type, or a video stimulus type.

In some embodiments, at least one session of the set of sessions may include the computing system providing a feedback indication for the user based on at least one of (i) a time elapsed between the presentation of the visual probe and a response by the user to presentation of the visual probe and (ii) a response by the user to the presentation of the visual probe.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a block diagram of a system for providing sessions to address chronic pain associated with conditions in users in accordance with an illustrative embodiment;

FIG. 2 depicts a block diagram for a process to select and present visual stimuli in accordance with an illustrative embodiment;

FIG. 3 depicts a block diagram for a process to select and present a visual probe corresponding to the visual stimuli, determine a time elapsed between a presentation of the visual probe and receipt of a response and to provide feedback in accordance with an illustrative embodiment;

FIG. 4 depicts a flow diagram of a method for providing sessions to address chronic pain associated with conditions in users in accordance with an illustrative embodiment;

FIGS. 5A and 5B depict screenshots of an example user interface for a system for providing sessions to address chronic pain associated with conditions in users in accordance with an illustrative embodiment;

FIGS. 6A and 6B depict screenshots of an example user interface for a system for providing sessions to address chronic pain associated with conditions in users in accordance with an illustrative embodiment;

FIG. 7 depicts a screenshot of an example user interface for a system for providing sessions to address chronic pain associated with conditions in users in accordance with an illustrative embodiment;

FIGS. 8A and 8B depict screenshots of an example user interface for a system for providing sessions to address chronic pain associated with conditions in users in accordance with an illustrative embodiment;

FIG. 9 depicts a screenshot of an example user interface for a system for providing sessions to address chronic pain associated with conditions in users in accordance with an illustrative embodiment;

FIGS. 10A and 10B depict a set of screenshots of an example user interface for a system for providing sessions to address chronic pain associated with conditions in users in accordance with an illustrative embodiment;

FIG. 11 depicts a flow diagram of a method of alleviating chronic pain associated with a condition in a user in need thereof, in accordance with an illustrative embodiment;

FIG. 12 depicts a timeline of a randomized, controlled, exploratory basket study to evaluate attention bias modification training in adults with chronic pain-related conditions in accordance with an illustrative embodiment;

FIG. 13 depicts a chart of a randomized, controlled, exploratory basket study to evaluate attention bias modification training in adults with chronic pain-related conditions in accordance with an illustrative embodiment; and

FIG. 14 is a block diagram of a server system and a client computer system in accordance with an illustrative embodiment.

### DETAILED DESCRIPTION

For purposes of reading the description of the various embodiments below, the following enumeration of the sections of the specification and their respective contents may be helpful:

Section A describes systems and methods for providing sessions to address chronic pain associated with conditions in users;

Section B describes methods of alleviating symptoms chronic pain associated with attention bias of users a condition in a user; and

Section C describes a network and computing environment which may be useful for practicing embodiments described herein.

A. Systems and Methods for Providing Sessions to Address Chronic Pain Associated with Conditions in Users

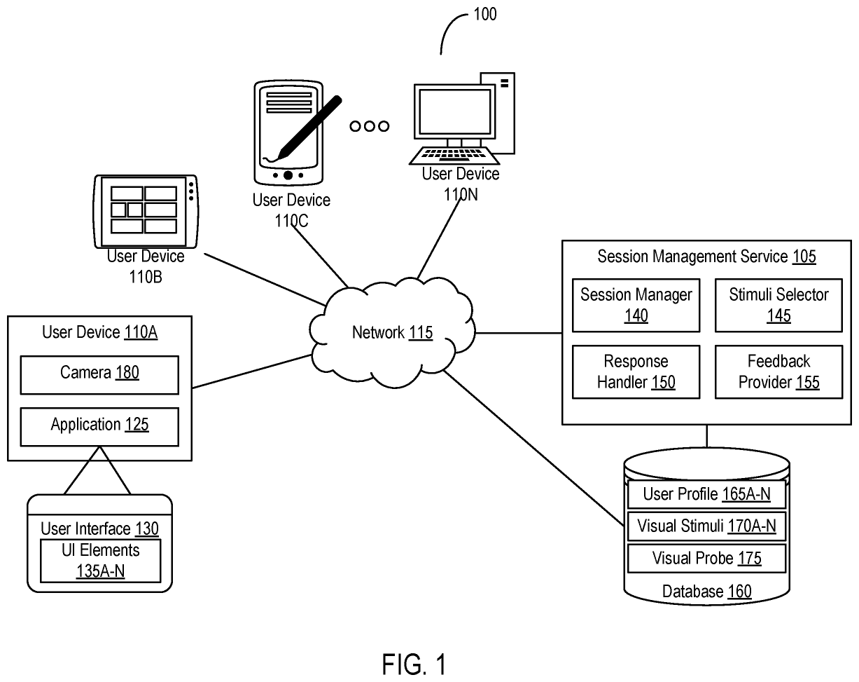

Referring now to FIG. 1, depicted is a block diagram of a system 100 for providing sessions to address chronic pain associated with conditions in users. In an overview, the system 100 may include at least one session management service 105 and a set of user devices 110A-N (hereinafter generally referred to as user devices 110), communicatively coupled with one another via at least one network 115. At least one user device 110 (e.g., the first user device 110A as depicted) may include at least one application 125. The application 125 may include or provide at least one user interface 130 with one or more user interface (UI) elements 135A-N (hereinafter generally referred to as UI elements 135). The session management service 105 may include at least one session manager 140, one stimuli selector 145, one response handler 150, or at least one feedback provider 155, among others. The session management service 105 may include or have access to at least one database 160. The database 160 may store, maintain, or otherwise include one or more user profiles 165A-N (hereinafter generally referred to as user profiles 165), one or more visual stimuli 170A-N (hereinafter generally referred to as visual stimuli 170) or a visual probe 175, among others. The functionality of the application 125 may be performed in part on the session management service 105. The functionality of the application 125 may also incorporate operations performed on the session management service 105, and vice-versa. For example, the application 125 can perform the functions of the stimuli selector 145, response handler 150, and the feedback provider 155 on the user device 110.

In further detail, the session management service 105 may (sometimes herein generally referred to as a computing system or a service) be any computing device comprising one or more processors coupled with memory and software and capable of performing the various processes and tasks described herein. The session management service 105 may be in communication with the one or more user devices 110 and the database 160 via the network 115. The session management service 105 may be situated, located, or otherwise associated with at least one server group. The server group may correspond to a data center, a branch office, or a site at which one or more servers corresponding to the session management service 105 is situated. The session management service 105 may be situated, located, or otherwise associated with one or more of the user devices 110. Some components of the session management service 105 may be located within the server group, and some may be located within the user device. For example, the session manager 140 may operate or be situated on the user device 110A, and the stimuli selector 145 may operate or be situated on the server group.

Within the session management service 105, the session manager 140 may identify a session to address chronic pain associated with a condition of the user, including a set of visual stimuli 170 to present to a user by the application 125 on respective user devices 110. The session manager 140 may identify a first visual stimulus associated with the chronic pain and a second visual stimulus neutral with respect to the chronic pain. The stimuli selector 145 may present the first and second visual stimulus during a first portion of the session relative to a fixation point on a display, such as the user interface 130. The stimuli selector 145 may remove the first and second visual stimuli from presentation on the display upon the elapse of the first portion. The stimuli selector 145 may present a visual probe corresponding to a position of the prior presented first stimulus or second stimulus to direct the user to interact with the visual probe during a second portion of the session. The response handler 150 may detect a response identifying an interaction associated with the visual probe and may determine a time elapsed between the presentation of the visual probe and the response. The feedback provider 155 may provide a feedback indication based on at least the elapsed time or the response.

The user device 110 (sometimes herein referred to as an end user computing device or client device) may be any computing device comprising one or more processors coupled with memory and software and capable of performing the various processes and tasks described herein. The user device 110 may be in communication with the session management service 105 and the database 160 via the network 115. The user device 110 may be a smartphone, other mobile phone, tablet computer, wearable computing device (e.g., smart watch, eyeglasses), or laptop computer. The user device 110 may include or be coupled with a camera 180. In some embodiments, the camera 180 may be disposed within the user device 110.

The camera 180 can be a camera or video capture device. The camera 180 may include multiple lenses or cameras to capture different fields of view relative to the camera. The camera 180 can capture images, frames, or pictures in one or more methods, such as point and shoot or image tracking. The camera 180 may detect motion, objects, people, edges, shapes, or various combinations thereof. In some embodiments, the camera 180 can be positioned to capture or detect an eye gaze of a user. An eye gaze of the user can refer to the direction, field of view, or focal point of the user's eyes. The eye gaze of the user can indicate where the user is focusing, concentrating, or viewing. The camera 180 can include one or more camera sensors to detect light to signal to the camera 180 to detect an eye gaze of the user. The camera 180, the session manager 105, the user device 110, or the application 125, among others, may perform various computer vision or other image processing operations on images captured by the camera 180.

The user device 110 may be used to access the application 125. In some embodiments, the application 125 may be downloaded and installed on the user device 110 (e.g., via a digital distribution platform). In some embodiments, the application 125 may be a web application with resources accessible via the network 115. The application 125 executing on the user device 110 may be a digital therapeutics application and may provide a session (sometimes herein referred to as a therapy session) to address symptoms associated with conditions. The user of the application 125 may be suffering from or at risk of a condition. The condition may include, for example, fibromyalgia (e.g., primary fibromyalgia, secondary fibromyalgia, hyperalgesic fibromyalgia, or comorbid fibromyalgia, among others), diabetic neuropathy (e.g., peripheral neuropathy, autonomic neuropathy, proximal neuropathy, or focal neuropathy, among others), rheumatoid arthritis (e.g., seropositive rheumatoid arthritis, seronegative rheumatoid arthritis, or palindromic rheumatism, among others), or IBS (e.g., with constipation, with diarrhea, or mixed, among others).

The attention bias may include, for example, avoidance of stimuli or an activity related to the chronic pain; chronic pain, mood, anxiety, or another reaction induced from stimuli associated with the symptom or the condition; or depression (or depressed mood), among others. The user may pay attention to stimuli which relate to symptoms of the condition, such as pain or actions which bring on symptoms, such as certain movements or behaviors. For example, the user may increase sensitivity to pain by refraining from movements that could cause pain, thereby further restricting the user and causing anxiety around the movement thought to cause pain. Other behaviors may cause or be related to a condition of the user. The application 125 may be used to present stimuli prompting the user to perform actions to reduce a bias towards negative stimulus associated with the condition of the user. The actions may be presented to the user as a result of sending a request to begin a session, detected measurements of the user received from the user device, or a scheduled time or period, among others.

The user may be at least partially concurrently taking medication to address the condition, at least partially concurrent with the sessions through the application 125. The medication may be at least orally administered, intravenously administered, or topically applied. For example, for rheumatoid arthritis, the user may be taking non-steroidal anti-inflammatory drugs (NSAIDs) (e.g., ibuprofen, naproxen, celecoxib, diclofenac, meloxicam, indomethacin), disease-modifying antirheumatic drugs (DMARDs) (e.g., methotrexate, sulfasalazine, leflunomide, adalimumab, etanercept, rituximab, abatacept, tocilizumab), Janus kinase (JAK) inhibitors (e.g., tofacitinib, baricitinib, upadacitinib), corticosteroids (e.g., prednisone, dexamethasone). For diabetic neuropathy, the user may be taking tricyclic antidepressants (TCAs) (e.g., amitriptyline, nortriptyline), selective serotonin-norepinephrine reuptake inhibitors (SNRIs) (e.g., duloxetine, venlafaxine), gabapentin, pregabalin, or lidocaine, among others. For fibromyalgia, the user may be taking duloxetine, milnacipran, pregabalin, amitriptyline, nortriptyline, or gabapentin, among others. For IBS, the user may be taking antispasmodics (e.g., dicyclomine, hyoscyamine), fiber supplements, laxatives (e.g., polyethylene glycol, lactulose, lubiprostone), anti-diarrheal medications (e.g., loperamide, bismuth subsalicylate, codeine phosphate), tricyclic antidepressants (e.g., amitriptyline, nortriptyline), or selective serotonin reuptake inhibitors (SSRIs) (e.g., fluoxetine, sertraline), among others. The application 125 may increase the efficacy of the medication that the user is taking to address the condition.

The application 125 can include, present, or otherwise provide a user interface 130 including the one or more UI elements 135 to a user of the user device 110 in accordance with a configuration on the application 125. The UI elements 135 may correspond to visual components of the user interface 130, such as a command button, a text box, a check box, a radio button, a menu item, and a slider, among others. In some embodiments, the application 125 may be a digital therapeutics application and may provide a session (sometimes referred to herein as a therapy session) via the user interface 130 for addressing a bias towards negative stimuli associated with the condition.

The application 125 can receive an instruction for presentation of the visual stimuli 170 or the visual probe 175 to the user. The visual stimuli 170 can be or include images or text to be presented via the user interface 130 and can be related to a negative association of the condition or not related to the condition. The visual probe 175 can be or include an action to be presented textually, as an image, as a video, or other visual presentation to the user and can include instructions for the user to perform the action to address symptoms associated with the condition.

An action related to the visual probe 175 can include interacting or not interacting with the user device 110. For example, the action can include pressing an image of the visual probe 175 presented by the user device 110. An image of the visual probe 175 can include a shape (e.g., circle, square), text, or image (e.g., of a face, of an object), among others. In some embodiments, performing the action indicated by the visual probe 175 can cause the application 125 to transmit a response indicating an interaction associated with the action to the session management service 105. The visual probe 175 can include instructions for the user to address the condition. For example, the visual probe 175 can include a message with instructions which describe the attention bias towards negative stimuli to be reduced. The visual probe 175 can include an interactive interface, through the user interface 130, to engage the user in one or more therapies designed to reduce or mitigate a bias towards negative stimuli associated with the condition. For example, the user may play a game on the user device 110 presented by the application 125 which incorporates one or more therapies to address the bias.

The database 160 may store and maintain various resources and data associated with the session management service 105 and the application 125. The database 160 may include a database management system (DBMS) to arrange and organize the data maintained thereon. The database 160 may be in communication with the session management service 105 and the one or more user devices 110 via the network 115. While running various operations, the session management service 105 and the application 125 may access the database 160 to retrieve identified data therefrom. The session management service 105 and the application 125 may also write data onto the database 160 from running such operations.

Such operations may include the maintenance of the user profile 165 (sometimes herein referred to as a subject profile). The user profile 165 can include information pertaining to a condition of a user, as described herein. For example, the user profile 165 may include information related to the severity of the condition, occurrences of the chronic-pain related condition, medications or treatments the user takes for the condition, and/or a duration of the condition, among others. The user profile 165 can be updated responsive to a schedule, periodically (e.g., daily, weekly), responsive to a change in user information (e.g., input by the user via the user interface 130 or learned from the user device 110), or responsive to a clinician (e.g., a doctor or nurse) addressing the user's condition, among others.

The user profile 165 can store and maintain information related to a user of the application 125 through user device 110. Each user profile 165 may be associated with or correspond to a respective subject or user of the application 125. The user profile 165 may contain or store information for each session performed by the user. The information for a session may include various parameters, actions, the visual stimuli 170, the visual probe 175, or tasks of previous sessions performed by the user, and may initially be null. The user profile 165 can enable streamlined communications to the user by presenting a task to the user which, based on at least the user profile 165, is most likely to aid the user in addressing symptoms of the user's condition or reducing the bias towards negative stimuli. This directed approach can reduce the need for multiple communications with the user, thereby reducing bandwidth and increasing the benefit of the user-computer interaction.

In some embodiments, the user profile 165 may identify or include information on a treatment regimen undertaken by the user, such as a type of treatment (e.g., therapy, pharmaceutical, or psychotherapy), duration (e.g., days, weeks, or years), and frequency (e.g., daily, weekly, quarterly, annually), among others. The user profile 165 may be stored and maintained in the database 160 using one or more files (e.g., extensible markup language (XML), comma-separated values (CSV) delimited text files, or a structured query language (SQL) file). The user profile 165 may be iteratively updated as the user provides responses and performs actions related to the visual stimuli 170, the visual probe 175, or the session, among others.

The visual stimuli 170 can be or include a stimulus or action to be presented textually, as an image, video, or other visual presentation to the user. For example, the visual stimuli 170 can include an animation to be presented via the user interface 130 of the user device 110. The visual stimuli 170 can include images such as photographs, digital images, art, diagrams, shapes, or other images. The visual stimuli 170 can include live, pre-recorded, or generated videos or animations, such as video recordings, animated shorts, or animated images (e.g., Graphics Interchange Format (GIF)). The visual stimuli 170 can include 3-dimensional (3D) visual presentations, such as holograms, projections, or other 3D visual media. The visual stimuli 170 can be in any size or orientation executable by the user interface 130. The visual stimuli 170 can include text, such as a word or sentence to be presented to the user via the user interface 130. The visual stimuli 170 can include instructions for the user to perform an action to address symptoms associated with the condition. For example, the visual stimuli 170 can include text or graphics which depict an action for the user to take or perform in relation to the visual stimulus 170.

The visual stimuli 170 can include two or more text-based or image-based stimuli. In some embodiments, the two or more stimuli can be presented during a first portion of the session at respective locations on the user interface 130. The visual stimuli 170 may be presented at locations relative to a fixation point presented on the user interface 130. In some embodiments, the visual stimuli 170 may be presented for a first portion of the session at their respective locations in relation to a fixation point. The fixation point can be a presentation of a point (e.g., a shape, image, text, or other such presentation) at a fixed location of the user interface 130. The fixation point may be located in the center of the user interface 130, the sides of the user interface 130, or in any location of the user interface 130. For example, the fixation point may be a fixed size circle presented at one location in the center of the user interface 130 for duration of the session.

While subsequent visual stimuli 170 of subsequent tasks or the same task of the session may be in different locations on the user interface 130, the location of the fixation point may remain the same for the duration of the session or task, despite a changing location of the stimuli for subsequent tasks. For example, two visual stimuli including text can be presented via the user interface 130. The two visual stimuli can be presented for a first portion of the session, each visual stimulus located in a respective location in relation to the fixation point. The user may focus on the fixation point, one or more of the visual stimuli 170, or a combination thereof, during the first portion. The one or more visual stimuli 170 may have a positive or neutral association and one or more other visual stimuli 170 may have a negative association with respect to the pain or condition. For example, a first visual stimulus can be a word or image with a negative association, such as the word “stabbing” or “shutting” or an image of a sad face. A second visual stimulus can be a word or image with a neutral or positive association, such as “love” or an image of a smiling face. In some cases, the first visual stimulus can be associated with the condition of the user. For example, the first visual stimulus can include a word associated with the condition, such as “pain” or an image or video associated with the condition, such as an image of someone in pain.

In addition, identifications of the visual stimuli 170 and the visual probe 175 may be stored and maintained on the database 160. For example, the database 160 may maintain the visual stimuli 170 or the visual probe 175 using one or more data structures or files (e.g., extensible markup language (XML), comma-separated values (CSV) delimited text files, joint photographic experts group (JPEG), or a structured query language (SQL) file). The visual probe 175 may prompt the user via the application 125 to perform an action via the application 125. For example, the application 125 may receive instructions to present two or more visual stimuli 170 to the user as a part of the session. Upon the elapse of a period of time, the visual stimuli 170 may be removed from presentation and the visual probe 175 may be presented at a location associated with one or more of the visual stimuli 170. The visual stimuli 170 and the visual probe 175 may be used to provide therapies to reduce the bias towards a negative stimulus associated with the condition, symptoms of the condition, or other cognitive or behavioral effects of the condition, or reduce the bias away from positive stimuli. The visual stimuli 170 and the visual probe 175 may be presented as games, activities, or actions to be performed by the user via the user interface 130. For example, the visual probe 175 may be presented after the presentation of the visual stimuli 170 to prompt the user to interact with the interface 130 if the visual probe 175 is not associated with a location of the negative visual stimulus 170.

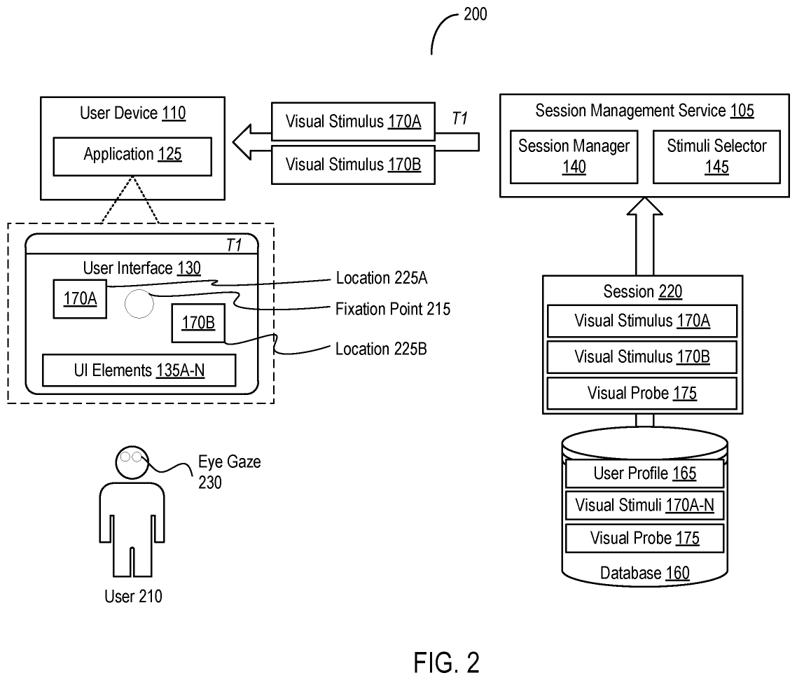

Referring now to FIG. 2, depicted is a block diagram for a process 200 to present the visual stimuli 170 and the visual probe 175 corresponding to the visual stimuli 170. The process 200 may include or correspond to operations performed in the system 100 to address chronic pain associated with conditions in users. Under the process 200, the session manager 140 executing on the session management service 105 may access the database 160 to retrieve, fetch, or otherwise identify the user profile 165 for a user 210 (sometimes herein referred to as a subject, patient, or person) of the application 125 on the user device 110. The user profile 165 may identify or define information associated with the user 210, the instance of the application 125 on the user device 110, and the user device 110, among others. For example, the user profile 165 may identify that user 210 has a certain bias towards negative stimuli, symptoms associated with a condition, or other cognitive or behavioral results from the condition. The user profile 165 may identify taking of medication by the user 210 to address the condition or associated symptoms of the condition in the user 210, or an indication of a value identifying a degree of association of a visual stimulus with chronic pain, among others.

The session manager 140 may determine or identify a session 220 for the user 210 to address chronic pain. The session 220 may correspond to, include, or define a set of visual stimuli to be presented to the user 210 via the application 125, such as the visual stimuli 170. Each visual stimulus 170 may be a visual stimulus to address the condition of the user. The visual stimuli 170 can be associated with the chronic pain or neutral with respect to the chronic pain. The session manager 140 can identify the session 220 to address chronic pain of the user 210 associated with the user profile 165.

The user profile 165 may include information on the visual stimuli 170, prior sessions (such as previous visual stimuli 170 identified for the user 210 or presented to the user 210), a performance associated with the visual stimuli 170 already identified for the user 210, a taking of medication by the user 210 to address the condition of the user, or an indication of a value identifying a degree of association of the corresponding visual stimulus with the chronic pain for the user 210, among others. The user profile 165 may also identify or include information on recorded performance of the bias, such as a number of occurrences of negative bias, symptoms associated with the condition, a number of occurrences of engaging in a bias towards negative, positive, or neutral stimuli associated with the condition, durations of prior occurrences, and taking of medication, among others. The user profile 165 may initially lack information about prior sessions and may build information as the user 210 engages in the session 220 via the application 125. The user profile 165 can be used to select the one or more visual stimuli 170 to provide via the application 125 to the user 210 in the session 220.

The session manager 140 may initiate the session 220 responsive to receiving a request from the user 210 via the application 125. The user 210 may provide, via the user interface 130 to execute through the application 125, a request to start a session. The request may include information related to the onset of the user's condition. The request can include attributes associated with the condition, such as an identification of the user 210 or the user profile 165, symptoms associated with the condition of the user 210, a time of the request, or a severity of the condition, among others. The application 125 operating on the user device 110 can generate the request to start the session 220 to send to the session management service 105 in response to an interaction by the user 210 with the application 125. In some embodiments, the session manager 140 may initiate the session responsive to a scheduled session time, responsive to a receipt of an indication of the value identifying a degree of association of a visual stimulus with the chronic pain, or based on the user 210 taking a prescribed medication to address the condition, among others.

In some embodiments, the session manager 140 can initiate the session responsive to the receipt of the one or more values each identifying a degree of association of a visual stimulus with the chronic pain for the user 210. Each value may identify a degree of association of a corresponding visual stimulus 170 with the chronic pain for the user 210, and can be a numeric value (e.g., a number between 0 to 1, −1 to 1, 0 to 10, −10 to 10, 0 to 100, and −100 to 100 ranging from less associated to more associated) or a binary value (e.g., 0 for not associated or 1 for associated), among others. The visual stimulus 170 may be initially part of a set of visual stimuli 170 potentially associated with chronic pain. For example, the set of visual stimuli 170 can be part of a word bank or a list of facial expressions pre-labeled as correlated with pain or the underlying condition. The user 210 can provide the value before, during, or subsequent to the session 220 provided by the session manager 140. In some embodiments, the user 210 can provide the value with the request to initiate the session 220. The user 210 can provide the value via the user interface 130. The user 210 can interact with the user interface 130 via the UI elements 135 to provide an input of the value identifying a degree of association of a corresponding visual stimulus with the chronic pain for the user 210, identification of the visual stimuli 170, a duration available for the session, or symptoms or conditions to be addressed during the session, among others. Upon entry, the session manager 140 can identify the value from the UI elements 135 on the user interface 130. In some embodiments, the values indicating the association between the chronic pain and the visual stimulus 170 can be stored as part of the user profile 165.

In some embodiments, the session manager 140 can use an eye gaze 230 of the user 210 to identify or determine the value indicating the degree of association of a corresponding visual stimulus 170 with the chronic pain for the user 210. The user interface 130 can present the visual stimulus 170 before, during, or subsequent to the session 220 provided by the session manager 140. The application 125 can monitor for or detect an eye gaze 230 of the user 210 using the camera 180 in combination with eye-tracking techniques (e.g., corneal reflection method, pupil-corneal reflex tracking, infrared eye tracking, machine learning-based algorithms). The eye gaze 230 can be or include a direction or position of the view of the user's eyes. The eye gaze 230 can include an orientation of the user's eyes. The eye gaze 230 can indicate where or at what the user 210 is looking. In some embodiments, the eye gaze 230 can indicate or correspond to a location on the user interface 130. The eye gaze 230 can indicate whether the user 210 looked at or viewed the visual stimuli 170 on the user interface 130. In some embodiments, the application 125 can also measure or determine a duration of the eye gaze 230 on the visual stimulus 170 on the user interface 130. The duration can identify a length of time that the eye gaze 230 of the user 210 is directed toward the visual stimulus 170 presented on the user interface 130.

Using the eye gaze 230 detected by the application 125, the session manager 140 can calculate or determine the value indicating the degree of association of a corresponding visual stimulus 170 with the chronic pain for the user 210. The session manager 140 can identify whether the eye gaze 230 is towards the visual stimulus 170 presented on the user interface 130 on the user device 110. The visual stimulus 170 presented can be pre-labeled as associated with the chronic pain in the word bank or a list of stimuli. If the eye gaze 230 of the user 210 is towards the visual stimulus 170 on the display, the session manager 140 can determine the value to indicate association of the corresponding visual stimulus 170 with the chronic pain. The session manager 140 can also determine the value based on a time duration of the eye gaze 230 towards the corresponding visual stimulus 170 relative to time duration of the eye gaze 230 for other visual stimuli 170 presented to the user 210. For example, the session manager 140 can set the value of the corresponding visual stimulus 170 higher than the value of another visual stimulus 170, when the time duration of the eye gaze 230 for the visual stimulus 170 is greater than the time duration of the eye gaze 230 for the other visual stimuli 170. Furthermore, the session manager 140 can set the value of the corresponding visual stimulus 170 lower than the value of another visual stimulus 170, when the time duration of the eye gaze 230 for the visual stimulus 170 is less than the time duration of the eye gaze 230 for the other visual stimuli 170. Conversely, if the eye gaze 230 of the user 210 is away from the visual stimulus 170 presented on the display, the session manager 140 can determine the value to indicate a lack of association of the corresponding visual stimulus 170 with the chronic pain. The session manager 140 can store the value for the visual stimulus 170 as part of the user profile 165.

The stimuli selector 145 executing on the session management service 105 may select or identify a set of visual stimuli 170 for presentation to the user 210 for the session 220. The stimuli selector 145 may select the visual stimuli 170 from the stimuli identified by the session manager 140. The stimuli selector 145 may select the visual stimuli 170 as a part of a session to perform attention bias modification training (ABMT) for the user 210 experiencing the condition. The set of visual stimuli 170 can include at least one visual stimulus 170 A associated with the condition. The visual stimulus 170A (herein also referred to as the first visual stimulus 170A) may be associated with chronic pain of the condition. As a part of the ABMT session 220, the stimuli selector 145 may select the first visual stimulus 170A and a second visual stimulus 170B for the user 210. The second visual stimulus 170B (also herein referred to as simply the visual stimulus 170B) may be neutral with respect to the chronic pain.

The first visual stimulus 170A can be a visual stimulus associated with the condition (e.g., condition-related, pain-related, or otherwise negatively associated). Conversely, the second visual stimulus 170B can be a visual stimulus not associated with the condition (e.g., neutral or positively associated). In some cases, the first visual stimulus 170A can be a negative stimulus associated with the condition. For example, the first visual stimulus 170A can include text containing a negative word associated with the condition, such as “pain,” “ache,” “fear,” or “tired.” The first visual stimulus 170A can include an image associated with the condition. For example, the first visual stimulus 170A can include an image of a sad or frowning face, an image of a stormy rain cloud, or an image of a snarling dog, among others. In some cases, the second visual stimulus 170B can be a positive or neutral stimulus. The second visual stimulus 170B may have no association with the condition. For example, the second visual stimulus 170B may include positive text containing one or more words such as “happy,” “good,” “smile,” or “love.” The second visual stimulus 170B can include neutral text containing one or more words such as “beach,” “puppy,” or “dinner.” The second visual stimulus 170B can include positive or neutral images. For example, the second visual stimulus 170B can be a picture of a tree, a baby, or a bicycle.

To select, the stimuli selector 145 may identify the set of visual stimuli 170 based on values identifying a degree of association between the respective visual stimulus 170 with the chronic pain of the user 210. By using the association values, the selection of the visual stimuli 170 can be more targeted at the particular association between each visual stimulus 170 and the chronic pain (or condition) formed in the mind of the user 210. The visual stimulus 170 can be selected based on a value identifying a degree of association with the chronic pain of the user 210. The value can be or include numeric values or scores, or descriptive indicators. The value can identify images, text, or other visual stimuli 170 which the user 210 associates with the condition, such as associating with chronic pain. The value may indicate visual stimuli 170 which the user 210 associates positively, or disassociates from the condition. For example, if the value is above a threshold value, the user 210 may associate a visual stimulus 170 with the chronic pain. The stimulus selector 145 can select the visual stimulus 170 associated with the chronic pain based on the value. Conversely, if the value is below the threshold value, the user 210 may not associate the visual stimulus 170 with the chronic pain, or the user 210 may associate the visual stimulus 170 with a positive or neutral stimulus. The stimulus selector 145 can select the visual stimulus 170 as not associated with the chronic pain based on the value, or can refrain from selecting.

The user can provide the value to the session manager 140, or the session manager 140 can retrieve the value. The user 210 can provide the value, or the session manager 140 can retrieve value from an external computing system, clinician, or library of pre-generated visual or auditory stimuli. The user profile 165 can include the value as a file, such as a comma-separated file (CSV), word document (DOC), standard MIDI file (SMF), or MP3, among others. The value can be provided via input into the application 125 operating on the user device 110. In some embodiments, the application 125 may present a user interface (e.g., via the user interface 130) prompting the user 210 to provide the value. The application 125 may present the UI elements 135 for the user to select, enter, or otherwise input the value. For example, the application 125 may present a sliding scale, series of questions, or text boxes associated with a visual stimulus 170 for the user to enter a value for a degree of association of the visual stimulus 170 with the chronic pain.

In some embodiments, the stimuli selector 145 or the session manager 140 may exclude a visual stimulus 170 from selection. A visual stimulus 170 may be excluded from selection based on the value. In some embodiments, if the value identifying a degree of association of the corresponding visual stimulus 170 is below a threshold value, the stimuli selector 145 may exclude the visual stimulus 170. In this manner, stimuli which the user 210 associates with the chronic pain can be more easily categorized as a stimulus related to the chronic pain or a stimulus neutral to the chronic pain, thereby providing customized stimuli selection for the user 210.

The session manager 140 may remove an excluded visual stimulus 170 from the database 160. The session manager 140 may remove the excluded visual stimulus 170 by deleting the visual stimulus 170 or otherwise moving the visual stimulus 170 from the database 160. In some embodiments, removing the visual stimulus 170 can cause the stimuli selector 145 to no longer be able to select the stimulus 170 for presentation during the session 220.

The session manager 140 may suspend usage of an excluded visual stimulus 170 for a period of time. The session manager 140 may suspend the excluded visual stimulus 170 from selection by the stimuli selector 145, from presentation by the application 125, or from usage by other various components of the system. The session manager 140 may determine the period of time for suspension of the excluded visual stimulus 170 based on the value indicating a degree of association of the visual stimulus 170 with the chronic pain. For example, a lower value (indicating less association of the visual stimulus with the chronic pain) may cause the session manager 140 to determine a longer suspension time than a suspension time associated with a higher value.

The visual stimuli 170 can include a type of visual stimuli. The type can correspond to the presentation of the visual stimuli. In some embodiments, the visual stimuli 170 can include a text image stimulus type, a scenic image stimulus type, a facial expression image stimulus type, or a video stimulus type, among others. A text image stimulus type can include or be related to a text, print, sentences, words, or fonts. The user 210 may associate certain text image stimulus types with the chronic pain, such as text reading “pain” or “hurt.” The user 210 may associate certain text image stimulus types not with the chronic pain or neutral to the chronic pain, such as “family,” “weather,” “fireplace,” or “beach,” among others. A scenic image stimulus type can include or be related to a visual stimulus which presents as an environment, scene, landscape, setting, or room, among others. The user 210 may associate certain scenic image stimuli as corresponding to the chronic pain or neutral to the chronic pain. For example, the user 210 may associate an image of a hospital as corresponding to the chronic pain, and an image of a beach as not associated with the chronic pain. A facial expression image stimulus type can include or be related to visual stimuli 170 of faces, emotions, moods, expressions, persons, emojis, or emoticons, among others. A video stimulus type can relate to or include a series of images or frames, a video, or an animation, among others.

In some embodiments, the stimuli selector 145 may select the visual stimuli 170 based on the type. The stimuli selector 145 may select a subsequent visual stimulus 170 for a session 220 based on the type of a previously presented visual stimulus 170. In some embodiments, the stimuli selector 145 may determine that a type of visual stimulus 170 is related to the user 210 based on the user profile 165. For example, the stimuli selector 145 may identify that a first type of visual stimulus elicited an interaction from the user 210 in a prior session more frequently than a second type of visual stimulus presented during the prior session. The stimuli selector 145 may select a visual stimulus for the session based on the types of visual stimuli presented during the prior session. In this illustrative example, the stimuli selector 145 may select the first type of visual stimulus for the session based on the first type of visual stimulus eliciting a higher interaction rate than the second type of visual stimulus during the prior session. Conversely, the stimulus selector 145 may select the second type of stimulus for presentation during the session over the first type of stimulus presented during the prior session to increase the difficulty of the session, or to promote the user 210 to recognize the visual stimulus of the second type.

The stimuli selector 145 may select the visual stimuli 170 based on the type during the prior session. The stimuli selector 145 may identify, from the user profile based on prior sessions, that the user 210 responds more quickly, responds more accurately, responds more consistently, or another metric related to the user's performance during the session, when the type of visual stimulus presented during the session is maintained, altered, or changed according to a pattern of types of stimuli. For example, the stimuli selector 145 may select the same type of stimulus as a previous session because a performance metric associated with the user profile 165 indicates that the user 210 increases one or more performance metrics when presented with the same type of visual stimulus. As another illustrative example, the stimuli selector 145 may select a different type of visual stimulus than presented during a previous session because a performance metric associated with the user profile 165 indicates that the user 210 increases one or more performance metrics when presented with a different type of visual stimulus. For example, a first user may historically (as recorded in the user profile 165) increase or not decrease her performance metric as related to the session 220 when presented with a text stimulus type, whereas a second user may historically increase or not decrease his performance metric as related to the session 220 when presented with alternating video and facial expression image stimulus types.

In some embodiments, the stimuli selector 145 may select the visual stimuli 170 based on the user profile 165. The user profile 165 may include historical information related to the user's condition, such as occurrences or types of symptoms, time of symptom occurrences, the intensity of the bias towards negative stimuli associated with the condition, demographic information, prescription information, location information, among others. For example, the session manager 140 may identify a visual stimulus 170 which has historically been positively associated by the user 210 towards improving the user's bias towards negative stimuli. For example, the session manager 140 may identify a visual stimulus 170 which the user 210 has indicated has a high degree of association with the user's chronic pain.

In some embodiments, the stimuli selector 145 may identify the visual stimuli 170 based on a session schedule. The session schedule may be determined by the session manager 140. In some embodiments, the session manager 140 may determine the session schedule based on a pre-defined session schedule, the user profile 165, or via an input from the user 210, a clinician associated with the user 210, or another outside input from an external computing system. The session manager 140 may define the session schedule based on historic sessions administered to the user 210. The session manager 140 may determine a session schedule based on a frequency of presentations of previous sessions or the visual stimuli 170, types of visual stimuli 170, or a performance metric associated with the user profile 165, among others.

The session schedule may define a frequency over a time period in which the user is to be provided with the session. In some embodiments, the frequency may be predetermined, such as at intervals of every hour, every day, or according to a pattern of frequency. In some embodiments, the frequency may be determined by the session manager 140. For example, the session manager 140 may determine a time of day at which the user 210 is most likely to access the application 125, respond to a visual probe 175, view the user interface 130, or experience chronic pain, among others and may generate or calculate a frequency based on its determinations. For example, the session manager 140 may identify that the user 210 most often accesses the application 125 in the morning and may establish the frequency of the sessions 220 to coincide with the morning. In some embodiments, the frequency of the sessions can be based on at least a clinician-sponsored frequency, or daily, weekly, or responsive to changes in a medication administered to address the condition with which the chronic pain may be associated.

The time period of the session schedule may be predetermined, such as by the user 210 or a clinician of the user 210. The user 210 may input a time period over which the sessions may be administered to the user 210. The user 210 may input a time at which a session 220 can be presented or administered via the application 125. The time period of the session schedule may be based on a performance metric of the user 210. In some embodiments, if the user 210 has a performance metric above a threshold metric, the session 220 may be a different duration than if the user 210 had a performance metric at or below the threshold metric. For example, if the user 210 is performing below a threshold metric, the session manager 140 may determine to extend the current session 220, or may determine that a subsequent session will have a longer duration.

In some embodiments, the stimuli selector 145 may identify the visual stimuli 170 based on a schedule of stimuli included in the session schedule. For example, the stimuli selector 145 may identify the first visual stimulus 170A to be a visual stimulus associated with the condition in accordance with the pre-defined schedule of stimuli. In this illustrative example, the stimuli selector 145 can identify a second visual stimulus 170B based on the subsequent stimulus of the pre-defined schedule. The session manager 140 may define a schedule or time at which the stimuli selector 145 may identify the visual stimuli 170 or at which to mark the visual stimuli 170 for presentation. In some embodiments, the stimuli selector 145 can identify the visual stimuli 170 based on a set of rules. The rules may be configured to provide a visual stimulus 170 or set of visual stimuli 170 to target the underlying causes or alleviate the chronic pain in the user 210 in a systematic, objective, and therapeutically effective manner. The rules may be based around time of presentation of a visual stimulus 170, time of an interaction with the user interface 130, the user profile 165, or other attributes of the system 100.

Upon identification, the session manager 140 may provide, send, or otherwise transmit the set of visual stimuli 170 to the user device 110. In some embodiments, the session manager 140 may send an instruction for presentation of the visual stimuli 170 via the user interface 130 for the application 125 on the user device 110. The instruction may include, for example, a specification as to which UI elements 135 are to be used and may identify content to be displayed on the UI elements 135 of the user interface 130. The instructions can further identify or include the visual stimuli 170. The instructions may be code, data packets, or a control to present the visual stimuli 170 to the user 210 via the application 125 running on the user device 110.

Continuing on, the instructions may include processing instructions for display of the visual stimulus 170 on the application 125. The instructions may include instructions for the user 210 to perform in relation to their session. For example, the instructions may display a message instructing the user 210 to take a medication associated with their session, or to focus on a fixation point on the user interface 130. The visual stimulus 170 may include a text, image, or video presented by the user device 110 via the application 125.

The application 125 on the user device 110 may render, display, or otherwise present the set of visual stimuli 170. The visual stimuli 170 may be presented via the one or more UI elements 135 of the user interface 130 of the application 125 on the user device 110. The presentation of the UI elements 135 can be in accordance with the instructions provided by the session manager 140 for presentation of the visual stimuli 170 to the user 210 via the application 125. In some embodiments, the application 125 can render, display, or otherwise present the visual stimuli 170 independently of the session management service 105. The application 125 may share or have the same functionalities as the session manager 140, the stimuli selector 145, or other components of the session management service 105 as discussed above. For example, the application 125 may maintain a timer to keep track of time elapsed since presentation of a previous visual stimuli 170. The application 125 may compare the elapsed time with a time limit for the visual stimulus 170. When the elapsed time exceeds the time limit, the application 125 may determine to present the visual stimuli 170. The application 125 may also use a schedule to determine when to present the one or more visual stimuli 170. The application 125 may present the visual stimulus 170 for display through the user interface 130 on the user device 110.

In some embodiments, the application 125 may display, render, or otherwise present the visual stimuli 170A and 175B for different time periods or concurrent time periods. The application 125 may present the first visual stimulus 170A for a first time period and the second visual stimulus 170B for a second time period. For example, the application 125 may present the first visual stimulus 170A during the first time period and then present the second visual stimulus 170B during the second time period. In some cases, the application 125 may delay the presentation of the second visual stimulus 170B after displaying the first visual stimulus 170A.

The application 125 can display, render, or otherwise present the visual stimuli 170A and 170B at an at least partially concurrent time. Presenting the visual stimuli 170A and 170B concurrently can refer to displaying the visual stimuli 170 during a concurrent time period, such as a first portion T1 of the session 220. A concurrent time period can refer to the first time period and the second time period overlapping in entirety or in part. For example, the presentation of the first stimulus 170A can overlap in duration with the presentation of the second stimulus 170B. The application 125 may present the visual stimuli 170A and 170B for the same period of time. For example, the application 125 can display the visual stimuli 170A and 170B during the first portion T1 of the session 220. In this manner, the display time of the first visual stimulus 170A and the second visual stimulus 170B can be the same as or equivalent.

The application 125 can display, render, or otherwise present the visual stimuli 170A and 170B at least partially concurrently with a fixation point 215. The visual stimuli 170 can be presented on a location of the user interface 130 which corresponds to the location of the fixation point 215. In some embodiments, the respective locations of the visual stimuli 170 are considered in relation to the fixation point. For example, a location 225A of the first visual stimulus 170A can be determined based on the fixation point 215, and a location 225B of the second visual stimulus 170B can be determined based on the fixation point 215. In some embodiments, the locations 225A and 225B or the fixation point 215 can be or include a discrete point or a perimeter enclosing the fixation point 215 or the locations 225A or 225B. The respective perimeters associated with the fixation point 215 or the location 225A or 225B may be any shape, such as a circle, square, polygon, or blob.

In some embodiments, the perimeters of the locations 225A or 225B may coincide with or include a perimeter or shape of the previously presented visual stimuli 170. For example, the perimeter of the first location 225A may include the area occupied by the presentation of the first stimulus 170A. For example, the perimeter of the second location 225B may be the same as the area occupied by the presentation of the second stimulus 170B. The locations 225A and 225B can be measured from the fixation point 215. The distance or position of the locations 225A and 225B in relation to the fixation point 215 can be measured by pixels, inches, or centimeters, among others. The distance between the fixation point 215 and any of the locations 225A or 225B can be measured from a center, perimeter, or point enclosed by the perimeter of the fixation point 215 or the locations 225A or 225B.

Upon the elapse of the first portion T1, the application 125 may cease presentation of the visual stimuli 170A and 170B. The elapse of the first portion T1 can be due to T1 exceeding a threshold period of time. The time for the first portion can range anywhere between 10 seconds to 3 minutes. For example, if T1 is greater than a threshold period of time for T1, the application 125 may stop presentation of the visual stimuli 170A and 170B. In some embodiments, the application 125 may stop presentation of the visual stimuli 170 responsive to an interaction by the user 210 with one or more of the UI elements 135. For example, the user 210 may select to stop presentation of one or more of the visual stimuli 170 during the execution of the application 125.

The application 125 may remove from presentation by the user interface 130 the first visual stimulus 170A, the second visual stimulus 170B, or both. The application 125 may stop presenting the visual stimuli 170 at any time. In some embodiments, the application 125 may stop presenting the visual stimuli 170 upon the elapse of the first portion T1. The application 125 may remove a subset of the visual stimuli 170 from presentation during the session 220. For example, the application 125 may remove the presentation of the first visual stimulus 170A and maintain the presentation of the second stimulus 170B. The application 125 may remove each visual stimuli 170 from presentation at different times. For example, the application 125 may remove the first visual stimulus 170A from presentation at a first time and the second visual stimulus 170B from presentation at a second time different from the first time.

The application 125 may remove the visual stimuli 170 from presentation while maintaining presentation of the fixation point 215. In some embodiments, the application 125 may continue to present the fixation point 215 when the first portion T1 elapses. For example, if the first portion T1 elapses, the application 125 may remove the visual stimuli 170 from display on the user interface 130 while maintaining the display of the fixation point 215 on the user interface 130. In this manner, the visual stimuli 170 can disappear from the display while maintaining the fixation point 215 on the user interface 130. Upon the removal of the visual stimuli 170 from display by the application 125, the stimuli selector 145 may select a visual probe directing the user 210 to interact with the visual probe.

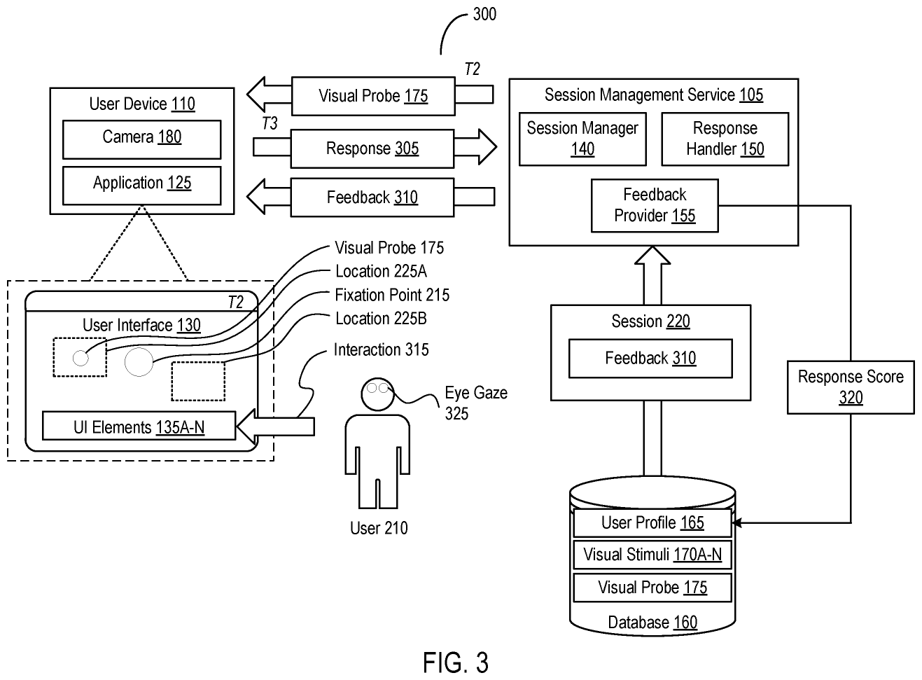

Referring now to FIG. 3, depicted is a block diagram for a process 300 to select and present a visual probe, determine a time elapsed between a presentation of the visual probe 175 and receipt of a response 305 and to provide feedback. The process 300 may include or correspond to operations performed in the system 100 or the process 200. Under the process 300, the stimuli selector 145 may select a visual probe 175 for presentation by the application 125. The response handler 150 may receive the response 305 indicating an interaction 205 by the user 210 with the visual probe 175. The response handler 155 may determine a time elapsed between the presentation of the visual probe 175 and the response 305. The feedback provider 155 may determine feedback 310 based on the elapsed time and the response 305. The session manager 140 may transmit the feedback 310 to the application 125 for presentation to the user 210.

The stimuli selector 145 may select a visual probe directing the user to interact with the visual probe 175. The stimuli selector 145 may select the visual probe 175 upon the removal of the visual stimuli 170 from presentation, upon the selection of the visual stimuli 170, upon commencement of the second portion T2 of the session 220, or at any time during the session 220. The second portion T2 can be immediately subsequent to the first portion T1 or at a delay, ranging between 100 ms to a few seconds. The visual probe 175 may be or include a visual presentation on user interface 130. The visual probe 175 may be or include any shape, image, video, character, or text to present upon the user interface 130. For example, the visual probe 175 may include a dot presenting on the user interface 130.

The stimuli selector 145 may identify or select the visual probe 175 upon or with the transmittal of the visual stimuli 170, the initiation of the session 220, the identification of the visual stimuli 170, or at another time of the session 220. In some embodiments, the stimuli selector 145 may identify the visual stimuli 170 for the first portion T1 and may identify the visual probe 175 for a second portion T2. The second portion T2 can range between 10 seconds to 3 minutes, and can correspond to the presentation of the visual probe 175 through the user interface 130.

In some embodiments, the stimuli selector 145 may determine, identify, or select one or more characteristics for the visual probe 175. The one or more characteristics of the visual probe 175 can include a location, a color, a size, a shape, an opacity, text (e.g., words, characters, or fonts), or other such characteristics of the visual probe 175. For example, the characteristic can include a green highlight over the visual probe 175 to indicate to the user 210 to select the visual probe 175. As another example, the characteristic can include text directing the user 210 as to a type of interaction 205 to perform, such as text denoting “Press the location of the neutral stimulus” or “Press the circle.” Each visual probe 175 can include different characteristics, such as different sizes, shapes, colors, or texts. For example, the stimuli selector 145 may select a blue circle as the visual probe 175 for one session, and a multicolored flower as the visual probe 175 for a different session.

The stimuli selector 145 may select one or more of the characteristics of the visual probe 175 based on a visual characteristic of the fixation point 215. The fixation point 215 can include visual characteristics similar to the visual characteristics described in conjunction with the visual probe 175. For example, the fixation point 215 can vary throughout sessions in size, shape, color, location, image, or opacity, among others. In some embodiments, the stimuli selector 145 may select the characteristic of the visual probe 175 based on the visual characteristics of the fixation point 215. For example, the stimuli selector 145 may select a circular visual probe 175 if the fixation point 215 is circular, or the stimuli selector 145 may not select a circular visual probe 175 if the fixation point 215 is circular. For example, the stimuli selector 145 may select a visual probe 175 that is a different color than the fixation point 215.

Upon selection of the visual probe 175 by the stimuli selector 145, the session manager 140 may transmit the visual probe 175 for presentation by the application 125. The session manager 140 may transmit the visual probe 175 during a second portion T2 of the session 220. The session manager 140 may transmit the visual probe 175 with the transmittal of the visual stimuli 170 during the first portion T1. In some embodiments, the session manager 140 may transmit the visual probe 175 upon the elapse of the first portion T1. The session manager 140 may transmit instructions with the visual probe 175 prompting the user 210 to interact with the visual probe 175.

The visual probe 175 may include instructions directing the user 210 to interact with the visual probe 175. The visual probe 175 can coincide with or include one or more of the UI elements 135. For example, the visual probe 175 can include a selectable icon on the user interface 130, or the visual probe 175 can indicate or be coupled with a button, slide, text box, or other such UI element 135. In some embodiments, the visual probe 175 can include instructions to interact with the visual probe 175 presenting on the user interface 130 via the UI elements 135. For example, an interaction 315 by the user 210 with the user interface 130 can include selecting the visual probe 175. The interaction 315 can include selecting one or more of the UI elements 135 associated with the visual probe 175. For example, the visual probe 175 may instruct the user 210 to press, touch, or actuate a UI element 135A.

The interaction 315 can include an action such as touching, pressing, or otherwise actuating a UI element 135 of the user interface 130 associated with the visual probe 175. For example, the user 210 can provide one or more interactions 315 through the application 125 running on the user device 110 by actuating one or more of the UI elements 135 as described herein. The user 210 can provide the interaction 315 by pressing a button associated with the application 125 and displayed via the user interface 130. In some embodiments, one or more first UI elements 135A can be associated with the visual probe 175. In this illustrative example, the user 210 can provide the interaction 315 associated with the visual probe 175 touching, tilting, looking at, or otherwise engaging with the first UI elements 135A.

The interaction 315 can include a series of actions performed sequentially or concurrently. For example, the interaction 315 can include a manipulation of the user device 110 and a pressing of a UI element 135. The manipulation of the user device 110 and the pressing of the UI element 135 can be performed concurrently as a part of the same interaction 315, or sequentially as a part of the same interaction 315. For example, the user 210 can tilt the user device 110 and press the UI element 135 at the same time, or the user 210 can tilt the user device 110 and then press the UI element 135. The application 125 may present one or more visual probes 175 via the user interface 130 to direct the user 210 to perform the interaction 315.

In some embodiments, the visual probe 175 may instruct the user 210 to tilt, turn, or otherwise manipulate the user device 110. For example, the visual probe 175 can instruct the user 210 to tilt the user device 110 towards a specified side of the user device 110, such as a left side of the user device 110. In some embodiments, the visual probe 175 may instruct the user 210 to direct an eye gaze 325 of the user towards a location of the user interface 130, such as the location 225A or the location 225B.

In some embodiments, the application 125 may display the visual probe 175 at or within the location 225A, the location 225B, or another location of the user interface 130. The application 125 may display the visual probe 175 at a location corresponding to a prior presentation of the visual stimuli 170. For example, the application 125 may display the visual probe 175 at the location 225B corresponding to the prior presentation of the second stimulus 170B. The location or presentation of the visual probe 175 can be disposed within the locations 225A or 225B. For example, the visual probe 175 may be fully or partially located, overlapping, or disposed within the location 225A associated with the first stimulus 170A. Likewise, the visual probe 175 may be fully or partially located, overlapping, or disposed within the location 225B associated with the second stimulus 170B. In this manner, the visual probe 175 can be associated with a prior presented visual stimulus based on the location of the prior presented visual stimulus and the current presentation location of the visual probe 175.

Presenting the visual probe 175 via the user interface 130 can include presenting the visual probe 175 according to a characteristic of the visual probe 175. In some embodiments, the application 125 can receive one or more of the visual characteristics of the visual probe 175 from the session manager 140. The application 125 may present the visual probe 175 according to those characteristics. The visual probe 175 may include visual characteristics related to an animation of the visual probe 175, duration of the presentation of the visual probe 175, location, size, shape, color, image, or other such visual characteristics of the visual probe 175. For example, the application 125 may present the visual probe 175 as a pulsing blue dot at location 225B on the screen pursuant to the visual characteristics of the visual probe 175.

The application 125 may monitor for at least one interaction 315 with the visual probe 175. The application 125 can monitor during the session 220 responsive to presentation of the visual stimuli 170, presentation of the visual probe 175, or responsive to receiving the interaction 220. The application 125 can monitor for receipt of the interaction 315. The application 125 can monitor for the interaction 315 through the user interface 130 or through sensors associated with the user device 110, among others. In some embodiments, the application 125 can monitor for the interaction 315 via the camera 180.

The application 125 may include eye-tracking capabilities to monitor for or detect the user 210 focusing on the visual probe 175 located at the location 225B. The eye-tracking capabilities can include object, line, motion, person, or other object detection, tracking, or recognition. The application 125 may perform the eye-tracking capabilities using the camera 180. In some embodiments, the camera 180 can detect light reflected off of the eyes of the user 210 to determine an orientation, focus, location, or direction of the user's eyes. For example, the camera 180 may detect an infrared light reflecting from the user's eyes and the application 125 may determine, based on the reflected infrared light, a location of the interface 130 that the user 210 is looking at. In some embodiments, the application 125 may access, actuate, or otherwise receive images or frames from the camera 180. The application 125 may identify, from the images or frames, the eye gaze 325, such as by an orientation of the eye relative to the fixation point 215. The application 125 may perform image processing in conjunction with, or as a part of, the eye-tracking capabilities. For example, the application 125 may identify, from the images of the camera 180, objects, lines, or persons.

The application 125 can receive multiple interactions 315 during a session. For example, the application 125 can monitor for a series of interactions 315 provided by the user 210 during the session. The application 125 may monitor and record information related to the received interactions 315. For example, the application 125 may monitor and record a time of an interaction 315, a duration of an interaction 315, a sequence of interactions 315, the visual stimulus 170 or the location 225A or 225B associated with the interaction 315, and/or the delay time between the presentation of the visual probe 170 and the interaction 315, among others. Upon detection of the interaction 315 with the user interface 130, the application 125 can identify whether the interaction 315 was on the location 225A or location 225B, or elsewhere.

Upon the user 210 providing the interaction 315, the application 125 may generate at least one response 305. The response 305 can identify the interaction 315. The response 305 can include the information about the interaction 315, such as a duration of the interaction 315, a time of the interaction 315, the location of the user interface 130 associated with the interaction 315, the visual stimulus 170 associated with the interaction 315, the visual probe 175 associated with the interaction 315 and/or a delay time between the presentation of the visual probe 175 and the interaction 315, among others. The application 125 can generate the response 305 for transmittal to the session management service 105. The response 305 can be in a format readable by the session management service, such as an electronic file readable by the session management service or data packets readable by the session management service 105, among others.

The response handler 150 can receive, identify, or otherwise detect the response 305. The response 305 can identify the interaction 315. The response handler 150 can receive the response 305 from the application 125. The response handler 150 can receive the response 305 at scheduled time intervals or as the interactions 315 occur during the session 220. For example, the response handler 150 can receive the response 305 during a portion T3 of the session 200, subsequent to the portion T2. The response handler 150 can query or ping the application 125 for the response 305. The response handler 150 can receive multiple responses 305 during a time period. For example, the response handler 150 can receive a first response 305 indicating a first interaction 315 and a second response 305 indicating a second interaction 315.

In some embodiments, the response 305 can include or identify the eye gaze 325. The application 125 can monitor for or detect an eye gaze 325 of the user 210 using the camera 180 in combination with eye-tracking techniques (e.g., corneal reflection method, pupil-corneal reflex tracking, infrared eye tracking, machine learning-based algorithms). The eye gaze 325 can be or include a direction or position of the view of the user's eyes. The eye gaze 325 can include an orientation of the user's eyes. The eye gaze 325 can indicate where or at what the user 210 is looking. In some embodiments, the eye gaze 325 can indicate or correspond to a location on the user interface, such as the location 225A or 225B. For example, the eye gaze 325 can indicate that the user 210 looked at the location 225B. The eye gaze 325 can indicate that the user 210 looked at or viewed the visual stimuli 170. For example, the eye gaze 325 can indicate that the user 210 looked at the visual stimulus 170B. In some embodiments, the application 125 can also measure or determine a duration of the eye gaze 230 on the visual stimulus 170 on the user interface 130. The duration can identify a length of time that the eye gaze 230 of the user 210 is directed toward the visual stimulus 170 presented on the user interface 130.

With the determination, the application 125 can generate the response 305 to include or indicate the eye gaze 325. In some embodiments, the response 305 can indicate the location, visual stimuli 170, or visual probe 175 that the user 210 looked at during the session 220. In some embodiments, the response 305 can include a time of the eye gaze 325 or a duration of the eye gaze 325. For example, the response 305 can indicate that the user 210 focused on the first visual stimulus 170A for 3 ms and the second visual stimulus 170B for 8 ms. The response 305 can indicate a pattern of the eye gaze 325. For example, the response 305 can identify that the eye gaze 325 switched between the first location 225A and the second location 225B at certain times, intervals, or a certain number of times. Upon generation, the application 125 can provide the response 305 including the identification of the eye gaze 325 to the response handler 150. In this manner, the response handler 150 can determine or identify the eye gaze 325 as being towards any of the visual stimuli 170, the visual probe 175, or the locations 225A or 225B or their respective corresponding visual stimuli.

The response handler 150 can store the response 305 including the interaction 315 in the database 160. The response handler 150 can store information related to the response 305, including a time of the response 305, actions associated with the interaction 315, the user profile 165 associated with the response 305, the visual probe 175 associated with the response 305, and the visual stimuli 170 associated with the response 305, among others. The response 305 may include or identify the interaction 315 by the user 210 with the visual probe 175. The response 305 may include a time for task completion. For example, the response 305 may include that the user spent 4 minutes to perform the action associated with the presentation of the visual probe 175.

The response 305 can include a total time for completion of the session 220 and may also include a time of initiation of the session 220 and a time of completion of the session. The response handler 150 may determine a time between the presentation of the visual probe 175 and the response 305. The response handler 150 can determine the time between the presentation of the visual probe 175 and the receipt of the response 305, the transmittal of the response 305, or the time of the interaction 315, among others. The response 305 may include the UI elements 135 interacted with during the duration of the presentation of the visual probe 175. For example, the response 305 may include a listing of buttons, toggles, or other UI elements 135 selected by the user 210 at specified times during the presentation of the visual probe 175. The response 305 may include other information, such as a location of the user 210 while performing the session, such as a geolocation, IP address, GPS location, or triangulation by cellular towers, among others. The response 305 may include measurements such as measurements of time, location, or user data, among others.

The feedback provider 155 can calculate, generate, or otherwise determine a response score 320 of the response 305 associated with the interaction 315 with the visual probe 175. The response score 320 can indicate a level of correctness or conversely a level of error associated with the response 305. A high response score 320 can correlate with a high level of correctness in selecting the location 225A of the prior-presented first neutral visual stimulus 170A. In this manner, a high response score 320 can correlate with an interaction 315 which does not relate to the bias towards the chronic pain. A low response score 320 can correlate with a low level of correctness (e.g., high level of error) in selecting the visual probe 175 which does not relate to the bias towards the condition. For example, a low response score 320 can relate to an interaction 315 with another location of the user interface 130 not associated with the neutral visual stimulus 170A or the visual probe 175, such as the location 225B or another location of the user interface 130. A low response score 320 can indicate that the user 210 is more likely to not select the visual probe 175.

In determining the response score 320, the feedback provider 155 may evaluate the response 305 based on the interaction 315. The response 305 may be correct, incorrect, or undeterminable. In some embodiments, the second visual stimulus 170B can be or include a neutral stimulus not associated with chronic pain of the user 210. Subsequent to the presentation of the visual stimuli 170 at the location 225B of the user interface 130, the application 125 may present the visual probe 175 at a third location associated with the second visual stimulus 170B, such as the location 225B. The user 210 may provide an interaction 315 related to the neutral visual stimulus 170B. For example, the user 210 may select the visual probe 175 by the application 125 using the UI elements 135. The user 210 may click, select, touch, or otherwise indicate a preference or selection for the visual probe 175 through the interaction 315. The interaction 315 may indicate the selection or preference for the second visual stimulus 170B associated with the visual probe 175.

The feedback provider 155 can identify or determine the response 305 by the user 210 as correct or incorrect based on the interaction 315 indicated in the response 305. The response 305 may be correct if the interaction 315 is associated with the second visual stimulus 170B or the visual probe 175B associated with the second stimulus 170B. The feedback provider 155 can determine the response 305 to be correct if the response 305 is associated with the interaction 315 corresponding to the visual stimulus 170B disassociating the user 210 from the chronic pain. The feedback provider 155 may identify the response 305 including the interaction 315 as correct.

The feedback provider 155 may identify the response 305 as correct if the interaction 315 indicates a bias towards a positive or neutral stimulus. In some embodiments, the interaction 315 can be associated with a positive or neutral visual stimulus 170B. For example, the interaction 315 can include selecting the visual probe 175 located in the location 225B of the prior presented positive or neutral visual stimulus 170B. The positive or neutral visual stimulus 170B can include positive or neutral imagery, text, or videos, among others, which is not related to the condition of the user 210 or to negative stimuli.

The feedback provider 155 may identify the response 305 as correct if the time between the presentation of the visual probe 175 and the response 305, as determined by the response handler 150, is below a threshold time. For example, the feedback provider 155 may determine the response 305 to be correct if the interaction 315 is performed by the user 210 within the threshold period of time. In some embodiments, the feedback provider 155 may determine that the response 305 is correct if the interaction 315 corresponds to the visual probe 175 and if the time between the presentation of the visual probe 175 and the response 305 is below a threshold period of time. In this manner, the user 210 can be trained to perform the tasks of the session 220 more quickly, thereby furthering their progress in redirecting biases away from stimuli associated with the chronic pain.

The feedback provider 155 may identify the response 305 as correct if the eye gaze 325 identified in the response 305 indicates a visual stimulus not associated with the chronic pain. In some embodiments, the feedback provider 155 may identify the response 305 as correct if the eye gaze 325 indicates towards the second visual stimulus 170B not associated with the chronic pain. In some embodiments, the feedback provider 155 may identify the response 305 as correct if the eye gaze 325 indicates towards the location 225B associated with the second visual stimulus 170B. In some embodiments, the feedback provider 155 may identify the response 305 as correct if a time associated with viewing the second visual stimulus 170B or the second location 225B is greater than a time associated with viewing the first visual stimulus 170A associated with the chronic pain or its corresponding location 225A. In some embodiments, the feedback provider may identify the response 305 as correct if the user 210 views the second visual stimulus 170B or its corresponding location 225B in a specified pattern as related to the other visual stimuli 170 or locations. For example, if the user 210 views the second visual stimulus 170B first and last during the presentation of the visual stimuli 170, the response 305 may be correct.

Conversely, the feedback provider 155 may identify the response 305 as incorrect if the interaction 315 is associated with the first stimulus 170A associated with the chronic pain. In some embodiments, the interaction 315 can be associated with a negative stimulus, a stimulus associated with the user's condition or chronic pain, or not with a neutral stimulus. For example, the interaction 315 can include selecting the location 225A associated with the negative visual stimulus 170A. In some embodiments, the interaction 315 corresponding to a location other than the location of the visual probe 175 associated with the neutral visual stimulus 170B can indicate an incorrect response 305. For example, the interaction 315 can including selecting any location not associated with the second visual stimulus 170B. The interaction 315 can include selecting a location of the user interface 130 above a threshold distance from the visual probe 175. For example, the interaction 315 can include a selecting a location above a threshold distance from the presentation of the visual probe 175, based on the fixation point 215. The threshold distance can correspond to a relative distance (e.g., at least 1 or 2 cm away) from the fixation point 215 at which the interaction 315 is to be determined correct or incorrect. In some embodiments, the feedback provider 155 may identify the response 305 as incorrect if the eye gaze 325 is indicated as being towards the first visual stimulus 170A or its corresponding location 225A.

Based on whether the response 305 is correct or incorrect, the performance evaluator 155 may calculate, generate, or otherwise evaluate the response score 320 for the user 210 based on the interaction 315 associated with the response 305. For example, the feedback provider 155 can set the response score 320 for a given response 305 as “1” when correct and “−1” when incorrect. In some embodiments, the feedback provider 155 may identify a reaction time or a correctness of the user 210 in selecting the visual probe 175. For example, the feedback evaluator 155 may determine, from the response 305, that the user 210 is not performing the interaction 315 as prompted by the visual probe 175 or that the user 210 is not interacting with the user interface 130 within a threshold time. The threshold time may correspond or define an amount of time in which the user 210 is expected to make the interaction 315 with one of the visual stimuli 170 or the visual probe 175. The feedback provider 155 may determine the response score 320 based on the eye gaze 325 as identified by the camera 180 and the application 125. With the determination, the feedback provider 155 can modify or adjust the response score 320 using at least one of the response times compared to the threshold time or the eye gaze 325.

In some embodiments, the feedback provider 155 can calculate, generate, or otherwise determine the response score 320 related to a rate of correct responses by the user 210. The rate of correct responses can be or include the number of correct responses of a set of responses over a period of time. The feedback provider 155 may aggregate the set of responses 305 over the period of time. The feedback provider 155 may generate the response score 320 based on the rate of correct responses for the period of time. For example, the period of time can be 6 weeks, and the feedback provider 155 may determine that of 100 received responses from the user 210 over the 6-week period, 40 are correct. In this illustrative example, the rate of correct responses for the period of time would be 40%. In some embodiments, the period of time associated with the rate of correct responses can be associated with the time period associated with the session schedule, described herein.

In some embodiments, the feedback provider 155 can calculate, generate, or otherwise determine the response score 320 related to the likelihood of overcoming the bias towards negative stimuli. The likelihood of overcoming the bias towards negative stimuli can refer to, include, or be related to a probability that the user 210 will cease to pay mind to visual stimuli associated with the chronic pain. For example, if the user 210 succeeds in ignoring negative stimuli associated with the chronic pain each time negative stimuli are presented to the user 210 via the application 125, the user 210 can be said to have a 100% rate of overcoming the bias towards negative stimuli. The likelihood of overcoming the bias towards negative stimuli may include a threshold number of occurrences of the bias. For example, the feedback provider 155 may not determine the likelihood until a threshold number of occurrences of the negative stimuli has arisen, until a threshold number of interactions 315 have been provided by the user 210, or until a threshold number of sessions have been provided to the user 210. The feedback provider 155 may determine the likelihood of overcoming the bias towards negative stimuli based at least on selections of the UI elements 135 during the session, the interaction 315, the response 305, the user profile 170, or a time of the session 220, among others.

In some embodiments, the feedback provider 155 can calculate, generate, or otherwise determine the response score 320 related to the eye gaze 325 of the user 210. The eye gaze 325 can indicate an increase in ability to resist the bias towards negative stimuli. For example, the user's eye gaze 325 may more frequently indicate or indicate for longer periods of time the neutral visual stimulus 170B or the location 225B associated with the neutral stimulus 170B over subsequent sessions.

In conjunction, the feedback provider 155 may produce, output, or otherwise generate feedback 310 for the user 210 to receive via the application 125 operating on the user interface 130. The feedback provider 155 may generate the feedback 310 based on at least the response score 320, the user profile 165, the response 305, or the historic presentations of the visual stimuli 170. The feedback 310 may include text, video, or audio to present to the user 210 via the application 125 displaying through the user interface 130. The feedback 310 may include a presentation of the response score 320. The feedback 310 may display a message, such as a motivating message, suggestions to improve performance, a congratulatory message, a consoling message, among others. In some embodiments, the feedback provider 155 may generate the feedback 310 during the session 220 being performed by the user 210.

Based on whether the response 305 is correct or incorrect, the feedback provider 155 can generate the feedback 310. The feedback can provide positive reinforcement or negative punishment for the user 210 depending on the responses 305 from the user 210. When the response 305 is determined to be correct, the feedback provider 155 can generate the feedback 310 to provide positive reinforcement. To provide positive reinforcement, the feedback provider 155 can generate a positive message, provide instructions for playback of positive sounds by the user device 110, or provide a haptic response via the user device 110, among others. In some embodiments, the feedback provider 155 can generate the positive feedback 310 to provide to the user 210 based on the response score 320 being at or above a threshold score. For example, if the response score 320 associated with the user 210 for a session 220 is above the threshold score, the feedback provider 155 can generate the feedback 310 to provide to the user 210 to encourage the user or to provide positive reinforcement.

Conversely, when the response 305 is determined to be incorrect, the feedback provider 155 can generate the feedback 310 to provide positive punishment. To provide positive reinforcement, the feedback provider 155 can generate a negative or consolatory message, provide instructions for playback of negative sounds by the user device 110, or provide a haptic response via the user device 110, among others. In some embodiments, the feedback provider 155 can generate or select the feedback 310 indicating negative feedback to provide to the user 210 if the response score 305 is below the threshold score. The generation of positive or negative reinforcement can be used in conjunction with the ABMT session to reduce the user's bias towards negative stimuli associated with their condition.

With successive responses, or upon a single response 305, the feedback provider 155 can send, convey, or otherwise provide the feedback 310 to the user 210 through the application 125. The feedback provider 155 may transmit feedback 310, such as provided in the form of an audio file (e.g., MPEG, FLAC, WAV, or WMA formats) or as part of an audio stream (e.g., as an MP3, AAC, or OGG format) to the application 125 on the user device 110. In some embodiments, the feedback provider 155 may send, transmit, or otherwise present feedback 310 for presentation via the application 125 during the performance of the session 220 or subsequent to the receipt of the response 305. For example, the response score 320 may indicate that the user 210 performing in the session 220 is below a threshold correctness. The feedback provider 155 may generate feedback related to the low response score 320, such as a motivating message including the response score 320. The feedback provider 155 can transmit and present the feedback 310 via the application 125 operating on the user device 110.

With the determination of the response score 320 or the feedback 310, the stimuli selector 145 may modify the presentation of subsequent sessions based on the response score 320 or the feedback 310. The stimuli selector 145 may modify the presentation of the first stimulus 170A, the second stimulus 170B, a subsequent visual stimulus, the visual probe 175, the fixation point 215, or a combination thereof. The stimuli selector 145 can provide instructions to the application 125 for display of the visual stimuli 170, the visual probe 175, or the fixation point 215. The stimuli selector 145 or the application 125 may modify the presentation of the visual stimuli 170 during the presentation of the visual stimuli 170 or subsequent to the presentation of the visual stimuli 170. For example, the stimuli selector 145 can modify the presentation of a first visual stimuli 170A as it is presented on the user interface 130 by the application 125. For example, the stimuli selector 145 can modify the presentation of subsequent visual stimuli 170N during the same session or a subsequent session.

The session manager 140 may modify the session schedule based on the response score 320 or the feedback 315. In some embodiments, the session manager 140 may modify the session schedule based on the rate of correct responses. The session manager 140 may modify the session schedule in duration, frequency, or the visual stimuli 170 presented or selected. For example, the session manager 140 may shorten the period of time associated with the session schedule if the rate of correct responses is above a threshold rate. For example, the session manager 140 may increase the frequency of the sessions for the session schedule if the rate of correct responses is below a threshold rate. Conversely, the session manager 140 may maintain or decrease the frequency of the sessions for the session schedule if the rate of correct responses is above the threshold rate. In this manner, the session manager 140 can generate a customized schedule based on the user's response score 320, responses 305, or the feedback 310.

The session management service 105 may repeat the functionalities described above (e.g., processes 200 and 300) over multiple sessions. The number of sessions may be over a set number of days, weeks, or even years, or may be without a definite end point. By iteratively providing visual stimuli and visual probes related to the neutral visual stimuli, based at least on the response score 320, user profile 165, responses 305, or the visual probe 175, the user 210 may be able to receive content to help alleviate the bias towards stimuli associated with chronic pain. This may alleviate symptoms faced by the user 210, even when suffering from a condition which could otherwise inhibit the user from seeking treatment or even physically accessing the user device 110. Furthermore, from participating in the session when presented through the user interface 130 of the application 125, the quality of a human computer interactions (HCI) between the user 210 and the user device 110 may be improved.

Since the visual stimuli 170 are more related to the user's condition (e.g., fibromyalgia, IBS, diabetic neuropathy, or rheumatoid arthritis, among others) and associated with symptoms arising from attention basis due to the condition, the user 210 may be more likely to participate in the session when presented via the user device 110. This may reduce unnecessary consumption of computational resources (e.g., processing and memory) of the service and the user device 110 and lower the usage of the network bandwidth, relative to sending otherwise ineffectual or irrelevant visual stimuli 170. Furthermore, in the context of a digital therapeutics application, the individualized selection of the visual stimuli 170 may result in the delivery of user-specific interventions to improve subject's adherence to the treatment. This may result in not only higher adherence to the therapeutic interventions but also lead to potential improvements to the user's condition and improved efficacy of the medication that the user is taking to address the condition.

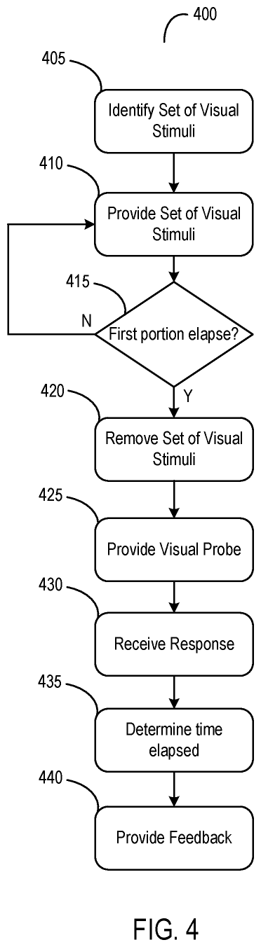

Referring now to FIG. 4, depicted is a flow diagram of a method 400 for providing sessions to address chronic pain associated with conditions in users. The method 400 may be implemented or performed using any of the components detailed herein, such as the session management service 105 and the user device 110, or any combination thereof. Under method 400, a computing system (e.g., the session management service 105, the user device 110, or a combination thereof) may identify a set of visual stimuli (405). The computing system may provide the set of visual stimuli to a client (e.g., the user device 110 or the application 125) (410). The set of visual stimuli may include the first visual stimulus 170A and the second visual stimulus 170B. In some embodiments, the set of visual stimuli can include a first visual stimulus corresponding to the chronic pain of the user and a second visual stimulus that is neutral in regard to the chronic pain. Providing the set of visual stimuli can include presenting the visual stimuli upon a display device associated with the computing system.

Upon presentation of the stimuli, the computing system may determine if the first portion of the session has elapsed (415). The computing system may determine if the first portion has elapsed by comparing a time period associated with the presentation of the visual stimuli to a threshold time period. If the computing system determines that the first portion of the session has not elapsed, the computing system may continue to provide the set of visual stimuli (410). If the computing system determines that the first portion has elapsed, the computing system may remove the set of visual stimuli (420). The computing system may remove the set of visual stimuli by providing instructions to remove the set of visual stimuli to the application, or by ceasing to provide instructions including the visual stimuli. Removing the set of visual stimuli can include removing the set of visual stimuli from presentation. In some embodiments, removing the set of visual stimuli can include removing the visual stimuli from the display device associated with the computing system. The computing system may maintain other presentations via the display with the removal of the set of visual stimuli from presentation. Upon or concurrent with removing the set of visual stimuli, the computing system may provide a visual probe (425).

The computing system may present the visual probe via the application executing on the computing system. The computing system can receive a response (430). The computing system can receive a response indicating the selection of the visual probe, a timing of the selection of the visual probe, or other information related to a selection. Upon receipt of the response, the computing system may determine the time elapsed (435). The computing system may determine the time elapsed between the presentation of the visual probe and the receipt of the response, the time elapsed between the presentation of the visual probe and the selection of the visual probe, or another time period. The computing system may provide feedback (440). The computing system may provide feedback based on at least the response or the time elapsed. The computing system may transmit the feedback for display via the application executing on the computing device. The computing system may display the feedback.

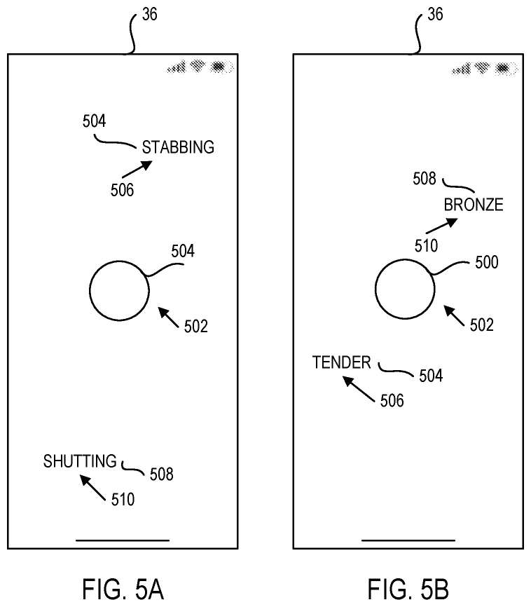

Referring now to FIGS. 5A and 5B, depicted are screenshots of a sample set of interfaces 36 for providing sessions to address chronic pain associated with conditions in users. FIG. 5A can include an interface 36. The interface 36 can be similar to or include the user interface 130. FIGS. 5A and 5B can include the application 125 installed thereon. The application 125 may present on a user interface or display such as the user interface 130 of the user device 110 or the interface 36. In some embodiments, the application 125 may present as a game, image, video, or interactive application 125 on the user device 110. FIG. 5A can depict the interface 36 including a central icon 500 located at a central position 502. The central icon 500 can be similar to or include the fixation point 215. FIG. 5A can include in the interface 36 a visual stimulus 504 at a location 506. The location 506 can correspond to or include the location 225A. The visual stimulus 504 can include text corresponding to a pain-related stimulus or a negative stimulus associated with the chronic pain of the user 210. FIG. 5A can include in the interface 36 a visual stimulus 508 at a location 510. The location 510 can be different than the location 506. The location 110 can correspond to or include the location 225B. The visual stimulus 508 can include text not corresponding to a chronic pain of the user 210, or text neutral to the condition or chronic pain of the user 210. FIG. 5B shows alternative positions of the visual stimuli 108 and 104 and their respective locations 510 and 506 in the interface 36. The depictions of the interface 36 shown in FIGS. 5A and 5B may occur during the first portion T1 of the session 220.

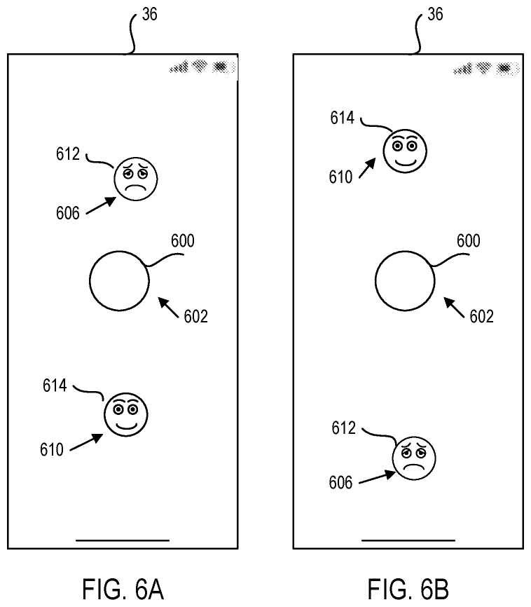

Referring now to FIGS. 6A and 6B, depicted are screenshots of a sample set of interfaces 36 for providing sessions to address chronic pain associated with conditions in users. The depictions of FIGS. 6A and 6B can be similar to or include functionality of the components of FIGS. 5A and 5B. FIGS. 6A and 6B can include the interface 36. FIGS. 6A and 6B can include the application 125 installed thereon. The application 125 may present on a user interface or display such as the user interface 130 of the user device 110 or the interface 36. In some embodiments, the application 125 may present as a game, image, video, or interactive application 125 on the user device 110. FIG. 6A can depict the interface 36 including the central icon 600 located at the central position 602. FIG. 6A can include in the interface 36 a visual stimulus 612 at the location 606. The visual stimulus 612 can include a depiction of a facial expression corresponding to a pain-related stimulus or a negative stimulus associated with the chronic pain of the user 210. FIG. 6A can include in the interface 36 a visual stimulus 614 at a location 610. The visual stimulus 614 can include a depiction of a facial expression not corresponding to a chronic pain of the user 210, or a facial expression neutral to the condition or chronic pain of the user 210. FIG. 6B shows alternative positions of the visual stimuli 614 and 612 and their respective locations 610 and 606 in the interface 36. The depictions of the interface 36 shown in FIGS. 6A and 6B may occur during the first portion T1 of the session 220.

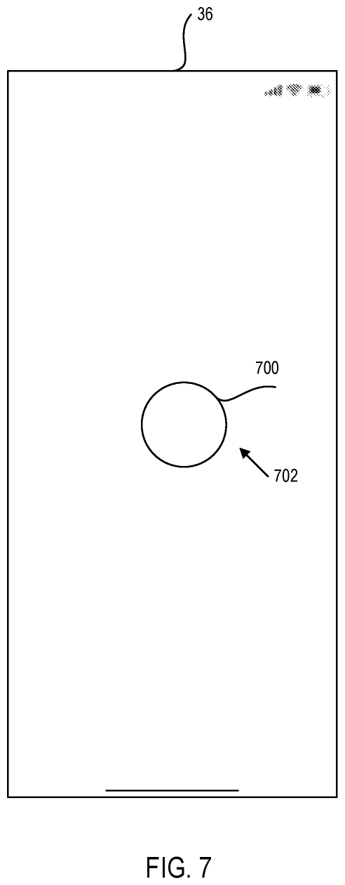

Referring now to FIG. 7, depicted is a screenshot of a sample interface 36 for providing sessions to address chronic pain associated with conditions in users. FIG. 7 can be similar to or include the functionality of the components of FIGS. 5A, 5B, 6A, and 6B. FIG. 7 can include the interface 36 depicting the central icon 700 at the location 702. In some embodiments, the depiction of the interface 36 in FIG. 7 can be presented by the application subsequent to the elapse of the first portion T1 or the removal of the presentation of the visual stimuli (e.g., the visual stimuli 170, 612, 614, 504, or 508, among others).

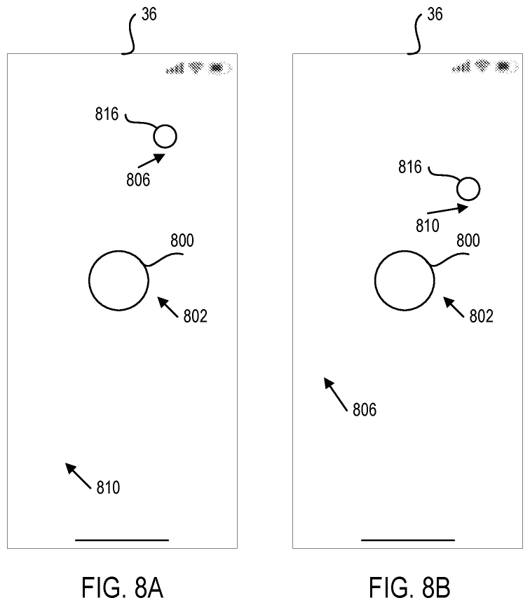

Referring now to FIGS. 8A and 8B, depicted is a set of screenshots of a sample interface 36 for providing a session to address chronic pain associated with conditions in users. FIGS. 8A and 8B can be similar to or include the functionality of the components of FIGS. 5A-7B. FIGS. 8A and 8B can include the interface 36 presenting the central icon 800 at the location 802. FIGS. 8A and 8B can depict the locations 806 and 810 without the presentation of their corresponding visual stimuli. FIGS. 8A and 8B can include a visual probe 816 depicted at the location 806. In this manner, the visual probe 816 can be placed at or within a threshold distance of a previously presented visual stimulus, such as the visual stimulus 806.

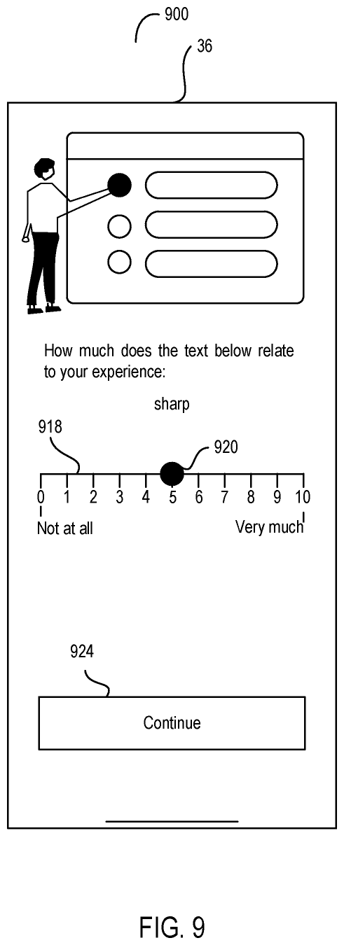

Referring now to FIG. 9, depicted is a screenshot of an example user interface for providing sessions to address chronic pain associated with conditions in users in accordance with an illustrative embodiment. FIG. 9 can include the user interface 36, a scale 918, and various UI elements 920, and 924. The scale 918 can be a psychometric scale. In some embodiments, the scale 918 can depict a range of symptoms, such as pain. For example, the scale 918 can depict a range of perceived pain for a user, such as the user 210. In some embodiments, the user can select, using the UI elements 920 on the scale 918, a value indicating a degree of association of a visual stimulus with the pain. The UI elements 920 and 924 can be similar to or include the UI elements 135. In some embodiments, the user can manipulate the UI elements 920 along the scale 918 to select the value indicating a degree of association of a visual stimulus with the pain. Upon selection of the value, the user may select the UI element 924 to continue with the session. The value selected by the user via the UI element 924 can be used to select visual stimuli 170 for the session.

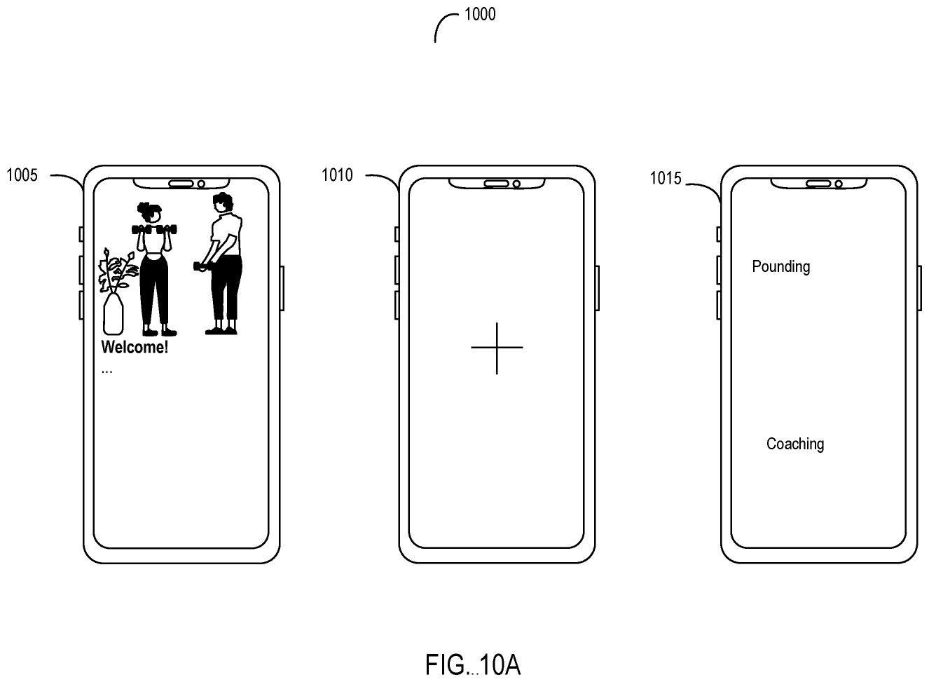

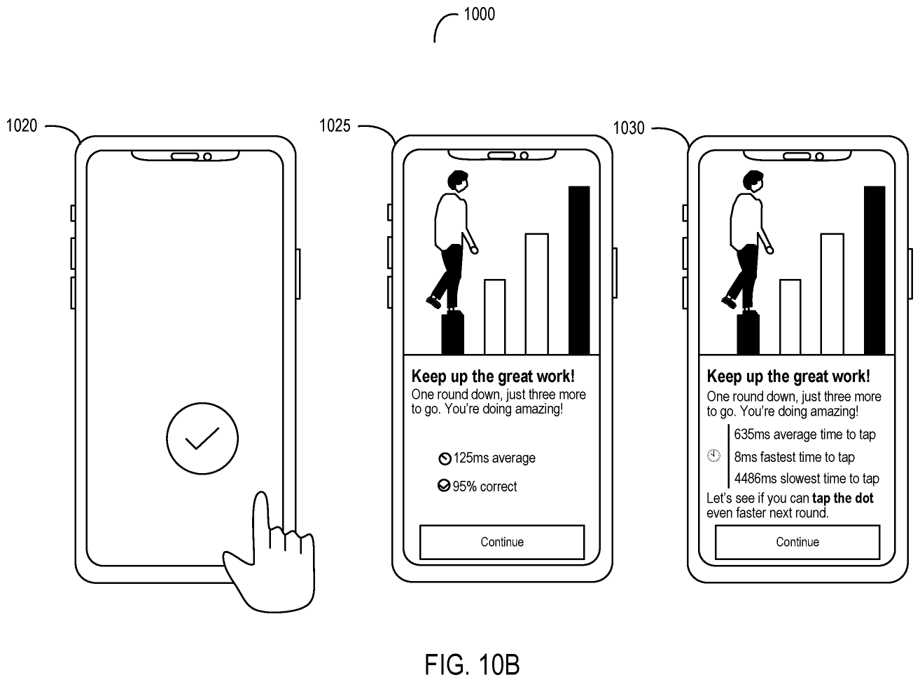

Referring now to FIGS. 10A and 10B, depicted is a set 1000 of screenshots of an example user interface for providing sessions to address chronic pain associated with conditions in users in accordance with an illustrative embodiment. The set 1000 can include screenshots 1005, 1010, 1015, 1020, 1025, and 1030. In some embodiments, the set 1000 can depict the systems and processes described herein with reference to FIGS. 1, 2, 3, and 4. In some embodiments, the screenshot 1005 can be or include an introductory interface for the session (e.g., the session 220). The screenshot 1010 can depict a presentation of two visual stimuli (e.g., the visual stimuli 170). The screenshot 1015 can depict a presentation of a fixation point (e.g., the fixation point 215). The screenshot 1020 can depict a visual probe (e.g., the visual probe 175). The screenshot 1025 can depict feedback identifying the average response time and the percentage of correct responses (e.g., the feedback 315). The screenshot 1030 can include feedback identifying the average response time, quickest time, and slowest time to respond (e.g., the feedback 315).

Since the application operates on the subject's mobile device, or at least a mobile device that she can access easily and reliably, e.g., according to the predetermined frequency (e.g., once per day), the application provides real-time support to the subject. For example, upon receiving a request from the user to initiate a session, the application initiates in real time, i.e., within at least a few milliseconds from receiving the request, a session. Such prompt guidance cannot be achieved via in-person visits, phone calls, video conferences or even text messages between the user and health care providers examining the user for Rheumatoid Arthritis, Diabetic Neuropathy, Fibromyalgia, or Irritable Bowel Syndrome (IBS). In this manner, the application is able to provide and customize tasks for the user based on the performance of the user. This can create an iteratively improving computing system (e.g., the service and the user's own device), thereby reducing overall consumption of computing resources and bandwidth and data communications with the increasing relevance of each stimuli. The filtering of visual stimuli for the user based on the user's indication that such stimuli are not related to the chronic pain related condition can also avoid a potential for the user to form new associations between these stimuli and the pain or underlying condition. Furthermore, the application can alleviate the chronic pain associated with the conditions apparent in the user as documented herein below.

B. Method of Alleviating Chronic Pain Associated with a Condition in a User

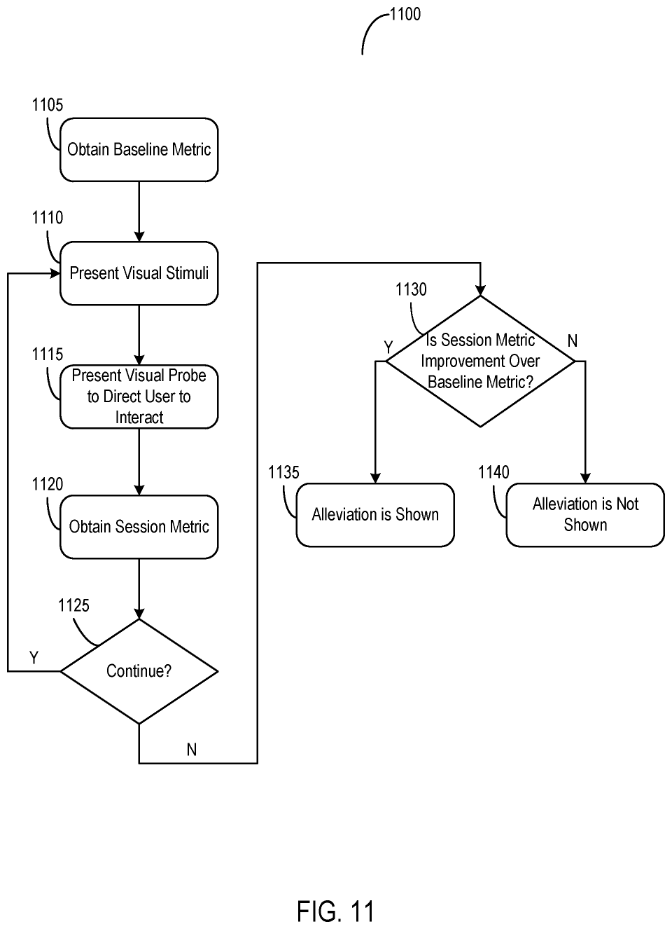

Referring now to FIG. 11, depicted is a flow diagram of a method 1100 of alleviating chronic pain associated with a condition in a user in need thereof. The method 1100 may be performed by any components or actors described herein, such as the session management service 105 and the user device 110, among others. The method 1100 may be used in conjunction with any of the functionalities or actions described herein in Section A or in the Examples in Section B. In brief overview, the method 1100 may include obtaining a baseline metric (1105). The method 1100 may include presenting a set of visual stimuli during a session (1110). The method 1100 may include presenting a visual probe to direct the user to interact (1115). The method 1100 may include obtaining a session metric (1120). The method 1100 may include determining whether to continue (1125). The method 1100 may include determining whether the session metric is an improvement over the baseline metric (1130). The method 1100 may include determining that alleviation is shown (1135). The method 1100 may include determining that alleviation is not shown (1140).

In further detail, the method 1100 may include determining, identifying, or otherwise obtaining a baseline metric prior to any session (1105). The baseline metric may be associated with a user (e.g., the user 210) at risk of, diagnosed with, or otherwise suffering from a condition. In some cases, the condition of the user may include fibromyalgia (e.g., primary fibromyalgia, secondary fibromyalgia, hyperalgesic fibromyalgia, or comorbid fibromyalgia, among others), diabetic neuropathy (e.g., peripheral neuropathy, autonomic neuropathy, proximal neuropathy, or focal neuropathy, among others), rheumatoid arthritis (e.g., seropositive rheumatoid arthritis, seronegative rheumatoid arthritis, or palindromic rheumatism, among others), or IBS (e.g., with constipation, with diarrhea, or mixed, among others). In some cases, the user may have been experiencing chronic pain due to the condition for at least three months prior to collection of the baseline metric.

The user may be on a medication to address the condition, at least in partial concurrence with the sessions. For rheumatoid arthritis, the user may be taking non-steroidal anti-inflammatory drugs (NSAIDs) (e.g., ibuprofen, naproxen, celecoxib, diclofenac, meloxicam, indomethacin), disease-modifying antirheumatic drugs (DMARDs) (e.g., methotrexate, sulfasalazine, leflunomide, adalimumab, etanercept, rituximab, abatacept, tocilizumab), Janus kinase (JAK) inhibitors (e.g., tofacitinib, baricitinib, upadacitinib), corticosteroids (e.g., prednisone, dexamethasone). For diabetic neuropathy, the user may be taking tricyclic antidepressants (TCAs) (e.g., amitriptyline, nortriptyline), selective serotonin-norepinephrine reuptake inhibitors (SNRIs) (e.g., duloxetine, venlafaxine), gabapentin, pregabalin, or lidocaine, among others. For fibromyalgia, the user may be taking duloxetine, milnacipran, pregabalin, amitriptyline, nortriptyline, or gabapentin, among others. For IBS, the user may be taking antispasmodics (e.g., dicyclomine, hyoscyamine), fiber supplements, laxatives (e.g., polyethylene glycol, lactulose, lubiprostone), anti-diarrheal medications (e.g., loperamide, bismuth subsalicylate, codeine phosphate), tricyclic antidepressants (e.g., amitriptyline, nortriptyline), or selective serotonin reuptake inhibitors (SSRIs) (e.g., fluoxetine, sertraline), among others. The user may be of any demographic or trait, such as by age (e.g., an adult (above age of 18), late adolescent (between ages of 18-24)) or gender (e.g., male, female, or non-binary), among others.

The user may have one or more chronic pain associated with an attention bias due to the condition. The user may also have other symptoms relevant to the condition such as fatigue, and emotion (e.g., depressed mood), among others. The pain caused by the condition may include pain resulting from fibromyalgia, diabetic neuropathy, IBS, or rheumatoid arthritis, among others. The attention bias may include, for example, avoidance of stimuli or an activity related to the symptom, chronic pain induced from stimuli associated with the condition, among others.

The baseline measure may be obtained (e.g., by a computing system such as the user device 110 or the session management service 105 or a clinician separately from the computing system) prior to the user being provided with any of the sessions via a digital therapeutics application (e.g., the application 125 or the Study App described herein). The baseline measure may identify or indicate a degree of severity of the pain associated with an attention bias due to the condition. Certain types of metrics may be used for the different conditions described herein. For both conditions, the baseline metric may include, for example, a Patient Reported Outcomes Measurement Information System (PROMIS) value (e.g., PROMIS-29), brief pain inventory inference (BPI-I) value, a pain catastrophizing scale (PCS) value, a global rating of change (GRC) value, a user experience questionnaire value, eye gaze, and computerized assessment values, among others. Certain types of metrics may be used for one of fibromyalgia, diabetic neuropathy, IBS, or rheumatoid arthritis. In some embodiments, the metrics can include baseline attention bias measured using the eye gaze or the user interaction with the prompt to indicate association between stimuli and the pain or the condition.

The method 1100 may include identifying or selecting a set of visual stimuli (e.g., the visual stimuli 170) to present during a session (1110). The computing system (e.g., the application 125) may select the set of visual stimuli based on user input (e.g., a user input of a value identifying a degree of association of a corresponding visual stimulus with chronic pain), a response score (e.g., the response score 320) associated with a user profile (e.g., the user profile 170) and prior sessions (e.g., sessions 220) if any have been previously provided to the user. Using the values indicating degrees of association, the computing system can select and provide the set of visual stimuli more relevant to the user's personal association of the visual stimuli with the chronic-pain related condition. The visual stimuli may include text, images, or video, and may be selected in accordance with attention bias modification training (ABMT). The set of visual stimuli may include at least one visual stimulus associated with the condition (or the pain associated with the condition) and at least one other visual stimulus. The first visual stimulus (e.g., the first visual stimulus 170A) may be, for example, a pain-related visual stimulus, a condition-related visual stimulus, or otherwise negatively related visual stimulus, among others. The second visual stimulus (e.g., the second visual stimulus 170B) may be a neutral visual stimulus or a positive visual stimulus, among others.

With the selection of the set of visual stimuli, the computing system may present the first visual stimulus and the second visual stimulus on a display (e.g., the user interface 130). The computing system may present the first visual stimulus and the second visual stimulus at respective locations (e.g., the locations 225A and 225B) on the display in reference to a fixation point (e.g., the fixation point 215). The computing system may present the visual stimuli for a period of time, such as the first portion T1. Upon elapse of the period of time, the computer system may stop presenting the visual stimuli. The computer system may stop presenting the visual stimuli but may, in some embodiments, continue to present the fixation point.

The method 1100 may include presenting a visual probe to direct the user to interact (1115). The user may be prompted or directed (e.g., via the display) to perform at least one interaction (e.g., the interaction 315) with the visual probe (e.g., the visual probe 175) presented to the user. For instance, the computing system may display a shape, token, image, or other presentable UI element coupled with the visual probe or including the visual probe to prompt the user to interact with the display. The computing system may monitor for the interaction with the visual probe. The interaction may include looking at a location associated with the visual stimuli, a touch (e.g., a touch or click event) with the visual probe, among others. Upon detection, the computing system may identify (e.g., from the response 305) the visual probe of the set with which the user performed the interaction and a time of the interaction.

The method 1100 may include presenting, outputting, or otherwise providing feedback (e.g., the feedback 315). The computing system may generate the feedback to provide to the user based on the response. The computing system may determine whether the response is correct based on the interaction with the display upon the presentation of the visual probe, based on an elapsed time between the response and the presentation of the visual probe, or a combination thereof. When the response identifies the interaction was with the visual probe or within a threshold distance of the visual probe, the computing system may determine that the response is correct. When the response identifies an elapsed time between the response and the presentation of the visual probe to be under a threshold time period, the computing system may determine that the response is correct.

The method 1100 may include determining, identifying, or otherwise obtaining a session metric (1120). The session metric may be obtained (e.g., by the computing system such as the user device 110 or the session management service 105 or a clinician separately from the computing system) subsequent to the user being provided with at least one of the sessions via the digital therapeutics application. The session metric may identify or indicate a degree of severity of the symptom associated with an attention bias due to the condition of the user. The session metric may be of the same type of measurement as the baseline metric. Certain types of metrics may be used for the conditions described herein. For the conditions, the session metric may include, for example, a Patient Reported Outcomes Measurement Information System (PROMIS) value (e.g., PROMIS-29), brief pain inventory inference (BPI-I) value, a pain catastrophizing scale (PCS) value, a global rating of change (GRC) value, a user experience questionnaire value, eye gaze, and computerized assessment values, among others. Certain types of metrics may be used for one of Rheumatoid Arthritis, Diabetic Neuropathy, Fibromyalgia, or Irritable Bowel Syndrome (IBS). In some embodiments, the session metric can include attention bias measured using the eye gaze or the user interaction with the prompt to indicate association between stimuli and the pain or the condition.

The method 1100 may include determining whether to continue (1125). The determination may be performed by the computing system. The determination may be based on the set length (e.g., days, weeks, or years) of the trial or a set number of sessions to be provided to the user. For example, the set number of time instances may range between 2 to 8 weeks or 1 to 90 days, relative to the obtaining of the baseline metric. When the amount of time from the obtaining of the baseline metric exceeds the set length, the determination may be to stop providing additional sessions. The method 1100 may repeat from step 1110, with the selection of the set of visual stimuli for the next session. The presentation of visual stimuli for the subsequent session may be altered, changed, or otherwise modified based on the response in the current session.

The method 1100 may include identifying or determining whether the session metric is an improvement over the baseline metric (1130). The determination may be performed by the computing system. The improvement may correspond to an amelioration or an alleviation in the chronic pain experienced by the user. The alleviation may be determined (e.g., by the computing system or a clinician examining the user) to have occurred when the session metric is increased compared to the baseline metric by a first predetermined margin or when the session metric is decreased compared to the baseline metric by a second predetermined margin. The margin may identify or define a difference in value between the baseline and session metrics at which to determine that the user shows reduction in the chronic pain or severity thereof. Whether the alleviation is shown by increase or decrease may depend on the type of metric used to measure the user with respect to the condition or the chronic pain. The margin may also depend on the type of metric used, and may in general correspond to the difference in value showing noticeable difference to the clinician or user with respect to the chronic pain, or showing a statistically significant result in the difference in the values between the baseline and session metrics.

The method 1100 may include determining that an alleviation of a chronic pain has occurred (1135). The determination may be performed by the computing system. In some embodiments, the alleviation of the chronic pain may occur when the session PROMIS value is increased from the baseline PROMIS value by the first predetermined margin. In some embodiments, the alleviation in the chronic pain may occur when the session BPI value is decreased from the baseline BPI-I by the first predetermined margin. In some embodiments, the alleviation in the chronic pain may occur when the session PCS value is decreased from the baseline PCS by the first predetermined margin. In some embodiments, the alleviation in the chronic pain may occur when the session metric value is increased from the baseline metric value by the second predetermined margin, for a computerized cognitive assessment value.

The method 1100 may include determining that no alleviation in the chronic pain has occurred (1140). The determination may be performed by the computing system. In some embodiments, the alleviation in the chronic pain may not occur when the session PROMIS value is not increased from the baseline PROMIS value by the first predetermined margin. In some embodiments, the alleviation in the chronic pain may not occur when the session BPI-I value is not decreased from the baseline BPI-I by the first predetermined margin. In some embodiments, the alleviation in the chronic pain may not occur when the session PCS value is not decreased from the baseline PCS by the first predetermined margin. In some embodiments, the alleviation in the chronic pain may not occur when the session metric value is not increased from the baseline metric value by the second predetermined margin, for a computerized cognitive assessment value.

### Example: A Randomized, Controlled, Single-Blind Exploratory Basket Study to Evaluate Attention Bias Modification Training in Adults with Chronic Pain-Related Disorders

### 1. Protocol Summary

### 1.1 Synopsis

CT-100 (e.g., the application 125) is a platform that provides interactive, software based therapeutic components that may be used as part of a multimodal treatment in future software-based prescription digital therapeutics. One class of CT-100 components are Digital Neuro-activation and Modulation (DiNaMo™) components. DiNaMo components target key neural systems (including but not limited to systems related to sensory-, perceptual-, affective-, pain-, attention-, cognitive control, social- and self-processing) to optimally improve a participant's cognitive and mental health.

The purpose of the proposed study is to evaluate initial effects of the ABMT DiNaMo component (the Study App) on measures of pain, pain-related functioning, and mood in pain indications. Chronic pain is a transdiagnostic condition which manifests in patient populations with diverse underlying medical conditions such as Rheumatoid Arthritis, Irritable Bowel Syndrome, Fibromyalgia, and Diabetic Neuropathy. Results derived from this research could be used as components within future digital therapeutics.

### Schedule of Activities and Assessments

Descriptive statistics were used for the user experience as well as engagement and safety parameters.

Schedule of Activities and Assessments

| | Screening | | | | | | |
|---|---|---|---|---|---|---|---|
| | Period | Study Period | | | | | Follow-up |
| | Screening | Baseline | Week 1 | Week 2 | Week 3 | Week 4 | Week 5 |
| Study Day and | Day −7 to | Day 1 | Day 7 | Day 14 | Day 21 | Day 28 | Day 35 |
| Window | Day −1 | (+3 days) | (+3 days) | (+3 days) | (+3 days) | (+3 days) | (+3 days) |
| Remote Visit | X | X | X | X | X | X | X |
| Informed | X | | | | | | |
| Consent | | | | | | | |
| Demographics | X | | | | | | |
| Identity | X | | | | | | |
| Verification | | | | | | | |
| Inclusion/Exclusion | X | X | | | | | |
| Criteria | | | | | | | |
| Additional | X | | | | | | |
| indication- | | | | | | | |
| specific | | | | | | | |
| information (not | | | | | | | |
| I/E) | | | | | | | |
| PHQ-8 | X | | | | | | |
| PROMIS pain | X | | | | | | |
| intensity item^{c} | | | | | | | |
| Download & | | X | | | | | |
| Activate App | | | | | | | |
| Randomization | | X | | | | | |
| BPI^{f} | | X | | | | X | X |
| BPI Interference^{f} | | | X | X | X | | |
| PROMIS ® 29 + 2 | | X | | X | | X | X |
| Profile v2.1 | | | | | | | |
| (PROMIS-29) | | | | | | | |
| Daily Pain | | | X | | | | |
| Intensity (NRS, | | | | | | | |
| momentary pain | | | | | | | |
| intensity, and 24 | | | | | | | |
| hours recall)^{g} | | | | | | | |
| Pain | | X | | X | | X | |
| catastrophizing | | | | | | | |
| scale (PCS) | | | | | | | |
| Patient-reported | | | | | | X | |
| Global Rating of | | | | | | | |
| Change (GRC) | | | | | | | |
| Pain Self- | | X | | | | X | |
| efficacy | | | | | | | |
| Questionnaire | | | | | | | |
| (PSEQ) | | | | | | | |
| Pain Vigilance | | X | | | | X | |
| and Awareness | | | | | | | |
| Questionnaire | | | | | | | |
| (PVAQ) | | | | | | | |
| Indication- | | X | | | | X | |
| Specific | | | | | | | |
| Assessments ^{d} | | | | | | | |
| Computerized | | X | | X | | X | |
| cognitive | | | | | | | |
| assessment | | | | | | | |
| (optional Altoida | | | | | | | |
| app) | | | | | | | |
| Computerized | | X | | | | X | |
| performance | | | | | | | |
| measures: | | | | | | | |
| Pain-information | | | | | | | |
| processing | | | | | | | |
| (Millisecond) | | | | | | | |
| User Experience | | | | | | | |
| Questionnaire | | | | | | | |
| Optional User | | | | | | | i |
| Experience | | | | | | | |
| Interview | | | | | | | |
| Deactivate App^{e} | | | | | | X | |
| AE/SAE Review | X | X | X | X | X | X | X |
| Con Meds | X | X | X | X | X | X | X |

Abbreviations:
AE = adverse event(s);
BPI = Brief Pain Inventory;
ET = Early Termination;
Neuro-Quality of Life Item Bank v2.0 - Cognition Function - Short Form;
GRC = Global Rating of Change;
NRS = Numerical Rating Scale;
PCS = pain catastrophizing scale;
PGIC = Patients Global Impression of Change;
PHQ-8 = Patient Health Questionnaire-8;
PROMIS = Patient Reported Outcomes Measurement Information System;
PROMIS-29 = PROMIS-29 + 2 Profile v2.1;
PROMIS-CF = PROMIS Item Bank v2.0 - Cognitive Function-Short Form 8a;
PSEQ = Pain Self-Efficacy Questionnaire;
PVAQ = Pain Vigilance and Awareness Questionnaire;
SAE = serious adverse event(s)
Note:
Participant were e-mailed a weblink to complete the indicated assessment online within the specified window.
a. Virtual visits were administered by site staff via telephone or a video conference platform as necessary.
b. Baseline assessments may be completed after the Screening Visit.
^{c}7-day recall period.
^{d} See Appendix 1 for indication-specific assessments to be administered.
^{e}After completion of the intervention period, on Day 28 the respective app became inert. After Day 28 or upon study withdrawal, participants will not have continued access to the content provided by the App during Days 1 to Day 28.
^{f}Baseline and Week 4 are the full BPI assessment. Week 1, Week 2, and Week 3 are the BPI Interference subscale.
^{g}Daily average pain intensity (24-hour recall) and momentary pain intensity assessed through the Study App and Digital Control App.
h. Only Baseline Visit were conducted by phone/video call.
i. A subset of participants were asked to complete an optional User Experience Interview (approximately 30) with the sponsor product development team conducted via remote teleconference software.
j. Participant downloaded the Altoida app (optional) at the Baseline Visit. Downloading the Altoida app and completing Altoida assessments is optional; absence of these assessments were not considered a protocol deviation.

### 2. Introduction

DiNaMo components target key neural systems (including but not limited to systems related to sensory-, perceptual-, affective-, pain-, attention-, cognitive control, social- and self-processing) to optimally improve a patient's cognitive and mental health.

The Attention Bias Modification Training (ABMT) DiNaMo component aims to implicitly retrain attention processes. Chronic conditions, such as pain, have been associated with biased attention processes, whereby patients are more attentive and hypersensitive to pain-related stimuli. In ABMT, users are trained to ignore emotional/pain content and instead orient towards neutral content. As pain and anxiety are highly comorbid and share similar neurocircuit alterations, ABMT has the potential to assist in the treatment of chronic pain indications.

The purpose of the proposed study is to evaluate initial effects of a CT-100 ABMT DiNaMo component (the Study App) on measures of pain, pain-related functioning and mood in pain indications. Participants have primary indications associated with chronic pain. Results derived from this research could be used as components within future digital therapeutics.

The ABMT DiNaMo component is an exercise with the goal of retraining attention biases. Chronic pain patients are hypersensitive to pain-related content, which leads to a stronger focus on pain-related stimuli. ABMT retrains attention processing by both reducing attention towards pain content and by promoting cognitive flexibility to permit easier shifting to neutral content.

### Application

The CT-100 ABMT DiNaMo component uses implicit training to redirect attention processes. This can help participants both react less and more easily disengage from pain-related stimuli. It is likely that ABMT can redirect attentional biases present in rheumatoid arthritis, irritable bowel syndrome, fibromyalgia, diabetic neuropathy, and other chronic pain syndromes.

ABMT training consists of regular, challenging exercises. In the current study, a treatment regimen of daily 7-minute sessions over a period of 4 weeks were tested. The Study App will also include daily pain ratings.

Both groups used an App (Study App or Digital Control App). Randomization will determine which participant receives which App. Participants were blinded to the study hypothesis.

### 3. Objectives and Endpoints

The primary study objective is to estimate the effect size for changes in pain interference in the Study App intervention group compared to the Digital Control App group. The secondary study objectives are to estimate the effect size for changes in pain-related endpoints (pain intensity, pain experience, general QoL, mood and functioning), to explore the feasibility of remote digital ABMT training, including engagement and experience with the Study App in participants with chronic pain, and to explore changes in computerized performance measures in the Study App group compared to the Digital Control App group.

The exploratory objectives were to explore state effects of ABMT sessions on pain experience and intensity and to explore durability of treatment response.

TABLE 1
Study Endpoints

Primary Endpoint
Change in pain experience from Baseline to Week 4 as measured by the Brief Pain Inventory -
Interference subscale (BPI)
Secondary Endpoints
Change in pain catastrophizing from Baseline to Week 4 as measured by the Pain
Catastrophizing Scale (PCS)
Change in pain intensity from Baseline to Week 4 as measured by the pain intensity
ratings (PROMIS Pain Intensity item: Numerical Rating Scale [NRS]: 7 days recall)
Change in attention to pain from Baseline to Week 4 as measured by the Pain Vigilance
and Awareness Questionnaire (PVAQ)
Change from Baseline to Week 4 as measured by the Pain Self-efficacy Questionnaire
(PSEQ)
Participant-reported Global Rating of Change (GRC) score at Week 4
Change in mood-related symptoms from Baseline to Week 4 as measured by the Quality
of Life (PROMIS-29; PROMIS ® 29 + 2 Profile v2.1 [PROMIS-29])
Change in processing pain-related information from Baseline to Week 4 as measured by
computerized performance measures (Millisecond dot probe an implicit association
tasks)
Engagement with the assigned Study App as measured by metrics such as daily app
usage and daily time in the Study App
Changes from Baseline to Week 4 in indication-specific measures
Experience with the respective apps as assessed by the User Experience Questionnaire at
Week 4 and participant feedback captured in follow-up qualitative interviews
Exploratory Endpoints
Change in momentary pain (NRS) before an ABMT session compared to after an ABMT
session
Duration of effects at Week 5 as measured by BPI and PROMIS
Change in general cognitive performance during the intervention as measured by an app-
based computerized cognitive assessment measure (optional Altoida app) at Baseline,
Week 2, and Week 4

### 4. Study Design

### Overall Design

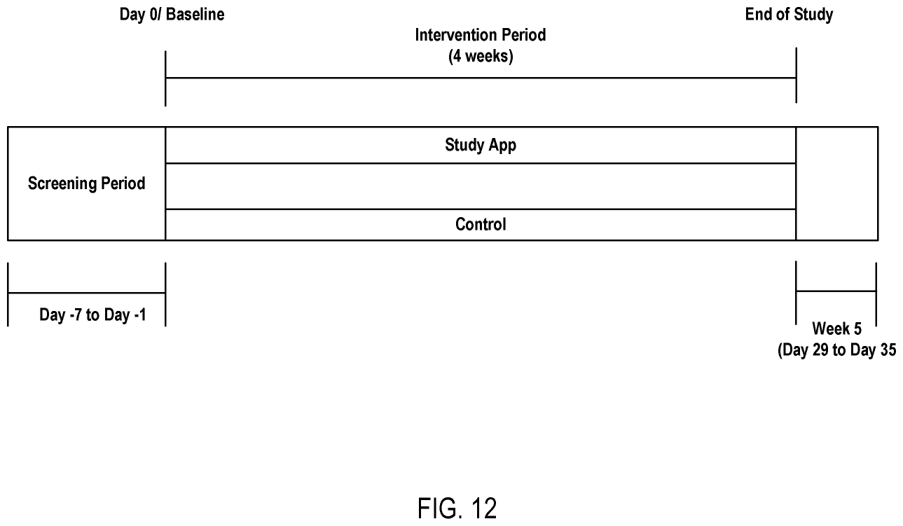

With reference to FIG. 12, this is a randomized, virtual study to evaluate the Study App as compared to the Digital Control App in participants with chronic pain-related disorders.

The study included an up to 1-week screening period, a 4-week intervention period and 1-week follow-up. Participants that meet eligibility criteria were enrolled in the study on Day 1.

The activities and assessments were completed according to the Schedule of Activities and Assessments. Study site staff implemented procedures remotely by telephone calls to participants and e-mailed weblinks to assessments. Participant engagement with their respective App (Study App/Digital Control App) were evaluated based on data captured within the app. Participants were also evaluated for adverse events and concomitant therapy use throughout the duration of the study through assessment prompts.

To mitigate participant expectation, participants in this trial were blinded to the efficacy hypothesis and their treatment assignment. Eligible participants were informed by trial staff that a) they will participate in the trial for up to 6 weeks (including the follow-up period) and were randomized to one of two digital therapeutic treatments and b) the purpose of the trial is to compare the effectiveness of these two digital therapeutic treatments. Both treatment arms were presented as possibly helping to improve chronic pain. No references to CT-100 or Digital Control should be made to the participant; both should only be referred to as the Study App.

Screening Period (Day −7 to Day −1): During a virtual screening visit, participants signed an electronic informed consent form (ICF), and all activities and assessments listed in the SoA were completed (Section 1.2). All eligible participants who have provided informed consent entered a screening period of up to 7 days to determine eligibility. Participants meeting eligibility requirements based on their online Screening Survey responses were provided a web link to schedule their Baseline Visit.

Screening and Baseline may occur on the same day if all required assessments have been completed per the protocol.

Baseline Virtual Visit (Day 1): Eligible participants were contacted for a Baseline Visit to review and confirm eligibility. Participants were considered eligible for study entry if they meet all inclusion and no exclusion criteria, based on investigator assessment.

Eligible participants were enrolled during a virtual study visit on Day 1. Participants were randomized 3:1 (Study App:Digital Control App). Assessments occurred according to the Schedule of Activities and Assessments (SoA).

Intervention Period (4 Weeks/Day 1 to Day 28): Site personnel assisted participants in downloading and installing their respective app onto their personal primary iPhone or Android smartphone. Upon enrollment, the Study App or Digital Control App were activated using unique login credentials. The process for activating and accessing the full therapeutic application during the baseline visit were the same for CT-100 and the Digital Control. This process is designed to minimize unblinding risk for the participant, and participants are considered enrolled upon randomization.

Participants were directed to access and perform tasks every day as directed by the respective App for 1-7 minutes per day, 7 days a week for 4 weeks. Assessments occurred according to the Schedule of Activities and Assessments (SoA).

Study App group: Participants utilized an app-based daily brain exercise (approximately 7 minutes) and tracked their daily pain intensity for approximately 1 minute a day, 7 days a week for 4 weeks.

Digital Control App group: Participants utilized an app to track their daily pain intensity approximately 1 minute 7 days a week for 4 weeks.

At the end of the treatment period, participants will complete the User Experience Questionnaire.

Follow-up Period (1 week/Day 29 to Day 35): Participants will complete follow-up assessments according to the SoA. A subset of participants were invited to complete an optional qualitative interview. Participants did not perform any activities within the Study App or the Digital Control App.

At the conclusion of a participant's participation in the study, participants were informed of the trial hypothesis (i.e., that one digital therapeutic was hypothesized to be more beneficial in improving chronic pain), but there was a need for a trial to confirm.

### Scientific Rationale for Study Design

This study is designed to evaluate the initial effects of the CT-100 ABMT component (the Study App) on pain interference and related outcomes.

Participants were assessed based on validated standard participant-rated outcomes. Participant engagement with the Study App were evaluated based on participant usage data captured within the Study App. Participants were also evaluated for safety throughout the duration of the study. The scales and assessments are described herein.

The study included a 7-day follow-up period to assess treatment durability, user experience, and medication use for all participants. In order to reduce bias, study participants were blinded to the hypothesis and treatment assignment, and informed that they received one of the two digital interventions being studied. The use of a comparator Digital Control poses minimal risk as all participants are maintained on their background therapy of SoC.

### End of Study Definition

The end of the study is defined as the date of the last contact, or the date of final contact attempt, for the last participant completing or withdrawing from the study. For the purposes of this study, participants who complete the assessments at Day 28 (+3) (Week 4) were defined as study completers.

### Inclusion Criteria

A participant were eligible for entry into the study if all of the following criteria are met:
1. Fluent in written and spoken English, confirmed by ability to read, understand, and sign the informed consent form.
2. Lives in the United States.
3. Adult between 18 and 65 years of age, inclusive, at the time of informed consent.
4. Meets indication-specific eligibility criteria (see Appendix 1), as reported by the study participant with adequate clinical documentation (to be provided to the study team upon request).
5. Self-reported average pain intensity during the last 7 days of ≥3 of 10 on the NRS scale associated with the primary indication.
6. Self-reported pain on at least 50% of days during the last week.
7. Has an active email address and is willing and able to receive and respond to email messages.
8. Has access to internet connection during the study duration.
9. Has an active PayPal account to receive study compensation, or is willing to create one.
10. Willing and able to comply with study protocol and assessments.
11. Is the sole user of an iPhone with an iPhone operating system (iOS) 14 or later or a smartphone with an Android operating system (OS) 10 or later for the duration of the study.
12. Is willing and able to receive Short Message Service (SMS) text messages and notifications on their smartphone.

### Exclusion Criteria

A participant were not eligible for study entry if any of the following criteria are met:
1. Has a comorbid acute pain condition, such as from current injuries.
2. Participation in a clinical trial (including psychotherapy, mindfulness, cognitive training, or drug treatment) within the last 2 months.
3. Initiation or change in primary disease-specific medication for 30 days prior to entering the study.
4. Daily use of opioids of 30 MME (Morphine Milligram Equivalents) or more.
5. Initiation or change in central nervous system (CNS)-active medication (e.g., antidepressants) during the last 2 months.
6. Planning to introduce new therapies or change therapies (including psychotherapy, mindfulness, cognitive training, or pharmacological treatment) during the study duration (1.5 months).
7. Self-reported substance use disorder within the past 1 year.
8. Visual, dexterity or cognitive deficit so severe that it precluded the use of an app-based reaction time-based activity, per investigator assessment.
9. Severe psychiatric disorder involving a history of psychosis (e.g., schizophrenia, bipolar disorder).
10. Has a Screening PHQ-8 score >20.
11. Psychiatric hospitalization in the past 6 months.
12. Other significant medical condition that, in the opinion of the Investigator, may confound the interpretation of findings to inform PDT development.

Lifestyle Considerations: Participants should have routine access to their smartphones for the duration of the trial.

Screen Failures: A screen failure is a participant from whom informed consent is obtained but who is not randomized or assigned trial intervention. Investigators must account for all participants who sign the informed consent documentation.

If a participant is found to not meet eligibility criteria for randomization into the study, the investigator completed the required Electronic Case Report Form (eCRF) pages. The primary reason for screen fail were recorded in the eCRF.

### Study Interventions

Study interventions are the Study App and a comparator Digital Control App (Table 2).

### Study Interventions Administered

Participants were administered with one of two study interventions by utilizing their assigned login credentials after randomization.

TABLE 2
Study Interventions

| | | |
|---|---|---|
| ARM Name | Study App | Digital Control App |
| Intervention Name | CT-100 | Digital Control |
| Type | Device | Device |
| Dose Formulation | Not applicable | Not applicable |
| Unit Dose Strength(s) | Not applicable | Not applicable |
| Dosage Level(s) | Not applicable | Not applicable |
| Route of | Not applicable | Not applicable |
| Administration | | |
| Use | Experimental | Comparator |
| IMP and NIMP | N/A | N/A |
| Sourcing | Click | Click |
| | Therapeutics, Inc. | Therapeutics, Inc. |
| Packaging and | Digital | Digital |
| Labeling | | |
| Current/Former | N/A | N/A |
| Name(s) or Alias(es) | | |

IMP = investigational medicinal product;
N/A = not applicable;
NIMP = non-investigational medicinal product

Study App (e.g., the application 125): The study intervention under evaluation is the CT-100 ABMT component, a digital mobile application. Participants randomized to this group downloaded and installed the Study App onto their own smartphone at the Baseline (Day 1) Visit and used the Study App daily for ABMT training and daily pain ratings (NRS) over the 4-week intervention period.

Digital Control App: Participants randomized to the control group downloaded and installed the Digital Control App onto their own smartphone at the Baseline Visit (Day 1) and used the app to complete daily pain ratings (NRS) over the 4-week intervention period in the Digital Control App.

App Download and Activation: During the Baseline Visit, site personnel assisted the participants randomized to download, install and activate their respective App. Instructions for installation and activation can be found in the Study App Instructions, provided separately. Only participants who are enrolled in the study may activate the apps. No App content was available prior to App activation following enrollment.

App De-Activation and Un-Installation: After completion of the intervention period (Day 28), the Study App and Digital Control App automatically de-activated and became un-usable for participants. Site personnel informed participants who complete the study or early terminate to uninstall their respective app.

Measures to Minimize Bias: Randomization and Blinding: Participants within each indication under study were randomly assigned in a 3:1 ratio to receive either the Study App or the Digital Control App. To mitigate participant expectation, participants in this trial were blinded to the efficacy hypothesis. This means that eligible participants were informed by trial site staff that a) they were randomized to one of two digital therapeutic treatments during the trial, and b) the purpose of the trial is to compare the effectiveness of these two digital therapeutic treatments, which may or may not improve chronic pain-related symptoms and experiences. No references to the “Digital Control App” or “Control App” should be made to the participant; both interventions should only be referred to as the Study App.

Study Intervention Compliance: Participants were told to use their respective App (Study App or Digital Control App) as instructed by the App. Compliance with this regimen was not defined for this study. However, the level of App engagement was measured.

Continued Access to Study Intervention after the End of the Study: After completion of the engagement period (Day 28), the apps became inert. After Day 28, participants did not have continued access to the content provided by the App during Days 1 to 28. Participants who terminated early had the app disabled.

Concomitant Therapy: Participants continued to use their prescribed therapies while enrolled in this study. Participants self-reported any changes to concomitant therapies through the end of the follow-up period.

### Study Assessments and Procedures

Study assessments and procedures, including their timing, are summarized in the SoA. Adherence to the study design requirements, including those specified in the SoA, is essential and required for study conduct. Protocol waivers or exemptions are not allowed. Every effort should be made to ensure that the protocol required assessments and procedures are completed as described. Study assessments are described below.

Study Assessments: The following assessment scales are used in this study at the times as provided in the SoA.

Screening Survey: The Screening Survey is a non-validated survey developed by Click Therapeutics describing the ABMT daily exercises and asking the participant to reflect on whether they are motivated and willing to commit to ˜1-7 minutes daily of app-delivered tracking and/or exercises for four weeks. The survey also includes questions on demographics, medical history, medications, eligibility criteria, and pregnancy status. This questionnaire is completed by the participant, and their commitment to the treatment regimen were verbally confirmed during eligibility review prior to randomization.

Brief Pain Inventory (BPI): The BPI is a self-report measure used for clinical trials. The BPI has 32 items to assess pain severity and interference using numerical ratings scales (NRS 0-10), pain location, pain medications, and amount of pain relief in the past 24 hours. This measure has had excellent test-retest reliability and internal consistency in chronic pain studies. This questionnaire was completed by the participant. It takes approximately five minutes to complete.

The BPI interference subscale has seven items, each item rated using a numerical rating scale (NRS 0-10). The BPI interference subscale aims to assess how much pain impacts daily functions. This measure is used for both acute and chronic pain conditions. This questionnaire was completed electronically by the participant using the standard 24 hours and additionally a 1-week recall period to optimally align with the study and PROMIS-29 recall period. It takes approximately one minute to complete.

Pain Catastrophizing Scale (PCS): The PCS is a reliable and valid 13-item self-report measure used to assess catastrophic thinking relating to pain and is intended for adults (ages 18-64). The PCS consists of 5-point Likert scales across 3 subscales: Rumination (4 items), Magnification (3 items), and Helplessness (6 items). The subscales can be scored separately, or they can be summed to provide a total score. This questionnaire was a survey completed electronically by the participant. It takes approximately five minutes to complete.

Pain Vigilance and Awareness Questionnaire (PVAQ): The PVAQ is a 16-item self-report questionnaire where patients rate their vigilance and awareness of pain. The PVAQ consists of a NRS 0-5 and is intended for adults (ages 18+). This questionnaire was completed electronically by the participant. It takes approximately five minutes to complete.

Pain Self-Efficacy (PSEQ): The PSEQ is a 10-item self-report questionnaire where patients rate their confidence in the ability to do daily activities at present despite their current level of pain. The PSEQ consists of a NRS 0-6 and is both reliable and consistent. This questionnaire was completed electronically by the participant. It takes approximately three minutes to complete.

PROMIS-29+2 Profile v2.1 (PROMIS-29): PROMIS-29 is part of the Patient Reported Outcomes Measurement Information System (PROMIS). PROMIS-29 is a short form assessment that contains four items from each of seven PROMIS domains (Mood, Physical Function, Pain Interference, Fatigue, Sleep Disturbance, and Ability to Participate in Social Roles and Activities) plus one pain intensity question (0-10 numeric rating scale). The PROMIS-29 is universal rather than disease-specific (i.e., can assess health from patients regardless of disease or condition) and is intended for adults (ages 18+). Scores are produced for all seven domains. The domains are assessed over the past seven days. The PROMIS-29 has been widely administered and validated in a range of populations and settings. This electronic questionnaire is completed by the participant. It takes approximately seven minutes to complete.

The PROMIS Pain Intensity item (Global07) is part of the PROMIS-29 and is a single NRS item that assesses pain intensity from 0 (no pain) to 10 (worst pain imaginable) with a 7-day recall period.

Daily Pain Intensity: Daily Pain Intensity (NRS, 24-hour recall period) were assessed in both Apps to support blinding to hypothesis and understanding additive effects of ABMT beyond pain tracking. Additionally, the Apps assessed momentary pain intensity before versus after the ABMT intervention, to assess state effects of the ABMT intervention.

Computerized Performance Measures: There were two computerized cognitive performance assessments: the dot probe task and the implicit association task. These cognitive assessments were conducted during the Baseline Visit through the Millisecond software Computerized Performance Measures.

Attentional Bias Dot Probe Task

In this task, a fixation point is displayed in the center of the screen. Following this, participants are presented with words or images from two categories: painful and neutral. One stimulus can appear above the fixation point, and the other may appear below. After a short time, the words disappear, and a probe stimulus is placed where one of the stimuli once was. The participant must respond with a response key based on the shape of the probe. Trials can be either congruent (pain stimulus and probe in same location) or incongruent (neutral stimulus and probe in same location). The outcome measures are proportion-correct and mean reaction time for the overall task, for all congruent tasks, and for all incongruent tasks. The bias index is calculated by subtracting mean latency of congruent from incongruent. Positive indicates bias towards painful words. The size of this number indicates the strength of attentional focus in that category. This web-based electronic assessment is completed by the participant and takes approximately 6 minutes to complete.

Implicit Association Task

In this task, participants categorize attributes (e.g., “neutral”; “pain”) and target items (e.g., “me”; “not me”) into predetermined categories with keystrokes. One key sorts the attribute into the category on the left (e.g., “me”) and other sorts to the right (e.g., “not me”). For the test, participants sort into paired categories (e.g., left: “pain” OR “me”; right: “neutral” OR “not me”). These pairings are swapped in the second block of the test (e.g., left: “pain” OR “not me”; right: “neutral” OR “me”). The primary outcome is the d-score, which is a value ranging from −1 to 1. More negative scores indicate a stronger preference for non-conforming pairings (e.g., preferring “pain” and “not me”). More positive scores indicate a stronger preference for conforming pairings (e.g., “pain” and “me”). Other outcomes include percent correct and proportion of response latencies <300 ms. This web-based electronic assessment is completed by the participant and takes approximately 3.5 minutes to complete.

Computerized Cognitive Assessment (Altoida): The Altoida app is a validated computerized cognitive assessment providing digital biomarker data for cognition and functional abilities, including 13 neurocognitive domains (spanning everyday function and cognition), which correspond to the major neurocognitive networks, such as complex attention and cognitive processing speed. Nearly eight-hundred (800) individual features, such as reaction time, speed, attention- and memory-based assessments, as well as every device sensor input (or lack thereof) through accelerometer, gyroscope, magnetoscope, camera, microphone, and touch screen are collected during augmented reality and motor tasks.

The assessment was completed by the participant via a downloaded app on their personal phone and takes 10 minutes to complete. Use of the Altoida app is optional; missed assessments were not considered a protocol deviation.

Global Rating of Change (GRC): The GRC is a self-reported, single item 10-point Likert scale used to assess the participant's rating of overall improvement with their indication after the study intervention. This item was completed electronically by the participant.

Patient Health Questionnaire-8 (PHQ-8): The PHQ-8 is an 8-item self-report measure to establish mood disorder diagnoses as well as grade mood symptom severity. This electronic scale is completed electronically by the participant.

User Experience Questionnaire (and Optional Qualitative Interview): The User Experience Questionnaire is a questionnaire developed by Click Therapeutics to understand participants' experience with the Study App during the intervention phase. The questionnaire asked questions related to the perceived enjoyment, challenges, and related user experience and did not contain questions related to clinical outcomes. This questionnaire was completed electronically by the participant. It takes approximately seven minutes to complete.

Additionally, a subset of participants may participate in phone or videoconference based qualitative user interviews. These interviews gathered additional information about the users' experience with the two apps, such as favorite app features, usability of the features, challenges related to the interventions, or any other feedback from regularly interacting with the apps.

### Sample Size Determination

Approximately 30 participants were enrolled to each indication under study and were randomized 3:1 (Study App:Digital Control App). This sample size should be sufficient to measure the effect size with relative precision.

### Results of the 4-Week Randomized, Exploratory Basket Study of Attention Bias Modification Training (ABMT)

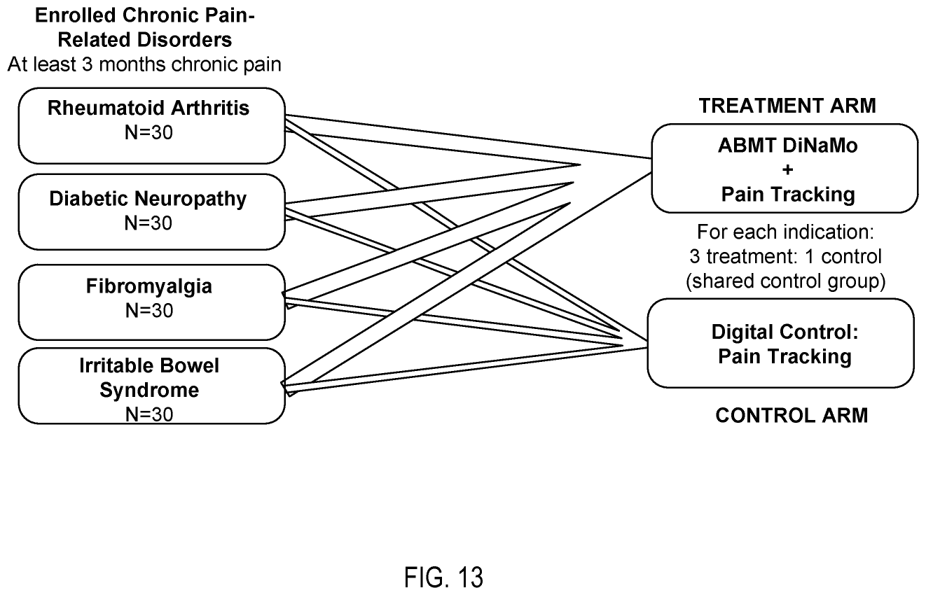

Referring now to FIG. 13, depicted is a chart of a randomized, controlled, exploratory basket study to evaluate attention bias modification training in adults with chronic pain-related conditions. The study was performed in accordance with the timeline laid out in FIG. 12. The ABMT (e.g., with personalized visual stimuli provided through the application 125) is developed to reduce hypersensitivity to pain-related information in chronic pain conditions. Some chronic pain conditions show altered attention processes, whereby patients are hypersensitive to pain-related information. Their attention is frequently drawn towards pain triggers, which significantly alters their experience of pain.

The ABMT intervention redirects biased attentional processes. ABMT asks the user to react to visual cues that are associated with neutral instead of triggering cues. This training retrains attention processes to be less captured by fear-inducing content and orient more easily and flexibly to neutral content. In this chronic pain ABMT, personal stimuli are used to divert attention away from pain towards neutral information. Stimuli are personalized to each patient's specific pain type. Brain Intervention Targets: Attention networks (Anterior Cingulate Cortex, parietal), pain-matrix/somatosensory (insula, limbic, S2).

Participants: Adults (22-65 years) with self-reported indication specific diagnosis, average pain intensity greater than or equal to 3 of 10 on the NRS scale during the last 7 days, and pain on at least 50% of days during the last week.

Interventions: Treatment (ABMT DiNaMo+Pain Tracking) vs. Digital Control (Pain Tracking).

Design: 3 treatment groups; 1 control group (share control group). The treatment groups can include participants in a group of chronic pain-related disorders who have experienced at least 3 months of chronic pain. These groups can include participants suffering from rheumatoid arthritis (N=30), diabetic neuropathy (N=30), fibromyalgia (N=30), and irritable bowel syndrome (N=30).

Endpoints: Pain, pain-related functioning, and mood.

Analysis: Pulled analysis—Treatment vs. Digital Control; within-group difference from baseline to week 4.

Supportive evidence for positive impact of ABMT on chronic pain

Pooled Within Group Analysis - Treatment vs Digital Control

| | Pooled Control | | Pooled Treatment | |
|---|---|---|---|---|
| | (n = 24) | | (n = 80) | |
| Pain Self Efficacy (Mean | −1.3 | (−5.5, 2.9) | 2.8 | (0.4, 5.2)*~ |
| Difference [95% CI]) | | | | |

Pain Catastrophizing Scale
(Mean Difference [95% CI])

| | | | | |
|---|---|---|---|---|
| Helplessness | 0.8 | (−0.9, 2.4) | −1.4 | (−2.2, −0.6)* |
| Rumination | 0.8 | (−0.6, 2.3) | −0.9 | (−1.7, 0.1)* |
| Total Score | 2.3 | (−1.4, 5.9) | −2.7 | (−4.5, 0.8)*# |

*p < 0.05
#indicates significant between group differences
~trending within group differences

Conclusion: Supportive evidence for the ABMT DiNaMo (e.g., the application 125) on pain related outcomes including pain related activity interference and the cognitive-emotional response to pain. Results suggest the ABMT DiNaMo may be a therapeutic intervention in chronic pain-related conditions.

Supportive Evidence for Positive Impact of ABMT on Pain Interference

Within Group Difference Baseline to Week 4

| | | | Rheumatoid | | | |
|---|---|---|---|---|---|---|
| | Pooled Control | | Arthritis | | Fibromyalgia | |
| | (n = 24) | | (n = 22) | | (n = 22) | |
| BPI-I (Mean | | | | | | |
| Difference [95% CI]) | | | | | | |
| 24-hour recall | −0.5 | (−1.5, 0.5) | −0.8 | (−1.5, −0.1)* | 0 | (−0.6, 0.6) |
| 7-day recall | −0.4 | (−1.4, 0.6) | −0.8 | (−1.3, −0.2)* | −0.2 | (−0.3, 0.6) |
| Pain Self Efficacy | −1.3 | (−5.5, 2.9) | 4.8 | (1.5, 8.2)* | 0.0 | (−4.1, 4.1) |
| (Mean Difference | | | | | | |
| [95% CI]) | | | | | | |
| Pain Catastrophizing | | | | | | |
| Scale (Mean | | | | | | |
| Difference [95% CI]) | | | | | | |
| Rumination | 0.8 | (−0.6, 2.3) | −0.6 | (−2.3, 1.0) | −1.4 | (−2.6, −0.2)* |
| Total Score | 2.3 | (−1.4, 5.9) | −2.2 | (−6.19, 1.8) | −2.6 | (−5.3, 0.2) |

*p < 0.05

Conclusions: Supportive evidence for the ABMT DiNaMo on self-reported pain measures was observed in rheumatoid arthritis, irritable bowel syndrome, and fibromyalgia. Significant reduction in pain interference observed in rheumatoid arthritis via BPI-I, with concurrent validation in PROM1S pain-intensity and self-efficacy ratings. Results suggest the ABMT DiNaMo may be a therapeutic intervention in certain pain-related conditions.

Supportive Evidence for ABMT in Pain-Related Disorders

Within-Group Difference Baseline to Week 4

| PROMIS-29 QoL | Pooled | | Rheumatoid | | Diabetic | |
|---|---|---|---|---|---|---|
| (Mean Difference | Control | | Arthritis | | Neuropathy | |
| [95% CI]) | (n = 24) | | (n = 22) | | (n = 18) | |
| Physical Function | −1.8 | (−4.18, 0.6) | 1.5 | (−0.5, 3.4) | 0.5 | (−1.1, 2.1) |
| Anxiety/Fear | 1.6 | (−1.1, 4.3) | −2.8 | (−5.97, 0.4) | −0.5 | (−3.9, 2.9) |
| Fatigue | 0.5 | (−0.6, 1.6) | −0.1 | (−1.3, 1.1) | −1.6 | (−2.8, −0.4)* |
| Sleep Disturbance | 0.1 | (−2.2, 2.3) | 1.3 | (−1.4, 4.0) | −2.2 | (−5.0, 0.7) |
| Social Participation | −1.0 | (−4.4, 2.4) | 2.2 | (0.2, 4.1)* | −0.2 | (−3.1, 2.7) |
| Pain Interference | 0.6 | (−2.4, 3.5) | −2.1 | (−4.7, 0.4) | 0.0 | (−3.6, 3.6) |
| Cognitive Function | −1.5 | (−5.4, 2.4) | −1.1 | (−5.2, 3.0) | −4.7 | (−10.3, 0.9) |

*p < 0.05

Minimal Important Change represents the threshold for which patients perceive themselves as importantly changed, typically reported to be between 2 and 6.

Conclusions: Domain improvement ≥minimally important change was observed in anxiety, social participation, and pain interference in the rheumatoid arthritis arm. Domain improvement ≥minimally important change was observed in social participation and pain interference in the irritable bowel syndrome arm. Results suggest the ABMT DiNaMo may meaningfully impact quality of life in certain indications.

### C. Network and Computing Environment

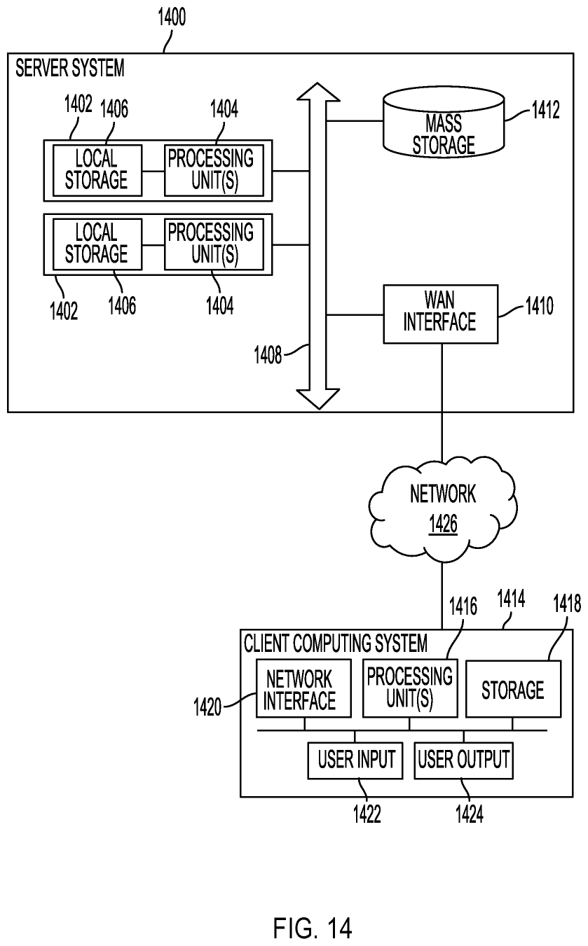

Various operations described herein can be implemented on computer systems. FIG. 14 shows a simplified block diagram of a representative server system 1400, client computer system 1414, and network 1426 usable to implement certain embodiments of the present disclosure. In various embodiments, server system 1400 or similar systems can implement services or servers described herein or portions thereof. Client computer system 1414 or similar systems can implement clients described herein. The system 1400 described herein can be similar to the server system 1400. Server system 1400 can have a modular design that incorporates a number of modules 1402 (e.g., blades in a blade server embodiment); while two modules 1402 are shown, any number can be provided. Each module 1402 can include processing unit(s) 1404 and local storage 1406.

Processing unit(s) 1404 can include a single processor, which can have one or more cores, or multiple processors. In some embodiments, processing unit(s) 1404 can include a general-purpose primary processor as well as one or more special-purpose co-processors such as graphics processors, digital signal processors, or the like. In some embodiments, some or all processing units 1404 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1404 can execute instructions stored in local storage 1406. Any type of processors in any combination can be included in processing unit(s) 1404.

Local storage 1406 can include volatile storage media (e.g., DRAM, SRAM, SDRAM, or the like) and/or non-volatile storage media (e.g., magnetic or optical disk, flash memory, or the like). Storage media incorporated in local storage 1406 can be fixed, removable, or upgradeable as desired. Local storage 1406 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1404 need at runtime. The ROM can store static data and instructions that are needed by processing unit(s) 1404. The permanent storage device can be a non-volatile read-and-write memory device that can store instructions and data even when module 1402 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1406 can store one or more software programs to be executed by processing unit(s) 1404, such as an operating system and/or programs implementing various server functions such as functions of the system 1400 or any other system described herein, or any other server(s) associated with system 1400 or any other system described herein.

“Software” refers generally to sequences of instructions that, when executed by processing unit(s) 1404, cause server system 1400 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1404. Software can be implemented as a single program or a collection of separate programs or program modules that interact as desired. From local storage 1406 (or non-local storage described below), processing unit(s) 1404 can retrieve program instructions to execute and data to process in order to execute various operations described above.

In some server systems 1400, multiple modules 1402 can be interconnected via a bus or other interconnect 1408, forming a local area network that supports communication between modules 1402 and other components of server system 1400. Interconnect 1408 can be implemented using various technologies, including server racks, hubs, routers, etc.

A wide area network (WAN) interface 1410 can provide data communication capability between the local area network (e.g., through the interconnect 1408) and the network 1426, such as the Internet. Other technologies can be used to communicatively couple the server system with the network 1426, including wired (e.g., Ethernet, IEEE 802.3 standards) and/or wireless technologies (e.g., Wi-Fi, IEEE 802.11 standards).

In some embodiments, local storage 1406 is intended to provide working memory for processing unit(s) 1404, providing fast access to programs and/or data to be processed while reducing traffic on interconnect 1408. Storage for larger quantities of data can be provided on the local area network by one or more mass storage subsystems 1412 that can be connected to interconnect 1408. Mass storage subsystem 1412 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1412. In some embodiments, additional data storage resources may be accessible via WAN interface 1410 (potentially with increased latency).

Server system 1400 can operate in response to requests received via WAN interface 1410. For example, one of modules 1402 can implement a supervisory function and assign discrete tasks to other modules 1402 in response to received requests. Work allocation techniques can be used. As requests are processed, results can be returned to the requester via WAN interface 1410. Such operation can generally be automated. Further, in some embodiments, WAN interface 1410 can connect multiple server systems 1400 to each other, providing scalable systems capable of managing high volumes of activity. Other techniques for managing server systems and server farms (collections of server systems that cooperate) can be used, including dynamic resource allocation and reallocation.

Server system 1400 can interact with various user-owned or user-operated devices via a wide-area network such as the Internet. An example of a user-operated device is shown in FIG. 14 as client computing system 1414. Client computing system 1414 can be implemented, for example, as a consumer device such as a smartphone, other mobile phone, tablet computer, wearable computing device (e.g., smart watch, eyeglasses), desktop computer, laptop computer, and so on.

For example, client computing system 1414 can communicate via WAN interface 1410. Client computing system 1414 can include computer components such as processing unit(s) 1416, storage device 1418, network interface 1420, user input device 1422, and user output device 1424. Client computing system 1414 can be a computing device implemented in a variety of form factors, such as a desktop computer, laptop computer, tablet computer, smartphone, other mobile computing device, wearable computing device, or the like.

Processing unit 1416 and storage device 1418 can be similar to processing unit(s) 1404 and local storage 1406 described above. Suitable devices can be selected based on the demands to be placed on client computing system 1414. For example, client computing system 1414 can be implemented as a “thin” client with limited processing capability or as a high-powered computing device. Client computing system 1414 can be provisioned with program code executable by processing unit(s) 1416 to enable various interactions with server system 1400.

Network interface 1420 can provide a connection to the network 1426, such as a wide area network (e.g., the Internet) to which WAN interface 1410 of server system 1400 is also connected. In various embodiments, network interface 1420 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 1422 can include any device (or devices) via which a user can provide signals to client computing system 1414; client computing system 1414 can interpret the signals as indicative of particular user requests or information. In various embodiments, user input device 1422 can include any or all of a keyboard, touch pad, touch screen, mouse or other pointing device, scroll wheel, click wheel, dial, button, switch, keypad, microphone, and so on.

User output device 1424 can include any device via which client computing system 1414 can provide information to a user. For example, user output device 1424 can include display-to-display images generated by or delivered to client computing system 1414. The display can incorporate various image generation technologies, e.g., a liquid crystal display (LCD), light-emitting diode (LED) display including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1424 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1404 and 1416 can provide various functionality for server system 1400 and client computing system 1414, including any of the functionality described herein as being performed by a server or client, or other functionality.

It will be appreciated that server system 1400 and client computing system 1414 are illustrative and that variations and modifications are possible. Computer systems used in connection with embodiments of the present disclosure can have other capabilities not specifically described here. Further, while server system 1400 and client computing system 1414 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of providing sessions to address chronic pain in users, comprising:
identifying, by a computing system, for a session to address chronic pain in a user, (i) a first visual stimulus associated with the chronic pain and (ii) a second visual stimulus being neutral with respect to the chronic pain;
presenting, by the computing system, relative to a fixation point on a display, the first visual stimulus at a first position and the second visual stimulus at a second position during a first portion of the session;
removing, by the computing system, from presentation on the display, the first visual stimulus and the second visual stimulus subsequent to elapsing of the first portion;
presenting, by the computing system, a visual probe corresponding to one of the first position or the second position relative to the fixation point, to direct the user to interact with the visual probe during a second portion of the session;
determining, by the computing system, a response by the user to presentation of the visual probe; and
providing, by the computing system, a feedback indication for the user based on the response by the user.

2. The method of claim 1, further comprising:
identifying, by the computing system, for each visual stimulus of a plurality of visual stimuli, an indication of a value identifying a degree of association of the corresponding visual stimulus with the chronic pain for the user based on at least one of: (i) an interaction with a user interface or (ii) an eye gaze with respect to the corresponding visual stimulus displayed on the user interface; and
wherein identifying the first visual stimulus further comprises selecting the first visual stimulus from the plurality of visual stimuli based on a corresponding value for the visual stimulus satisfying a threshold.

3. The method of claim 2, further comprising excluding, by the computing system from a plurality of visual stimuli, at least one visual stimulus for presentation to the user, responsive to a corresponding value of the at least one visual stimulus not satisfying the threshold.

4. The method of claim 1, further comprising:
determining, by the computing system, that the response by the user is correct, responsive to the user interacting with the visual probe where the second visual stimulus being neutral with respect to the chronic pain was presented on the display; and
wherein providing the feedback indication further comprises generating the feedback indication based on the determination that the response is correct.

5. The method of claim 1, further comprising:
determining, by the computing system, that the response by the user is incorrect, responsive to the user interacting on the display outside a threshold distance away from the where the second visual stimulus being neutral with respect to the chronic pain was presented on the display; and
wherein providing the feedback indication further comprises generating the feedback indication based on the determination that the response is incorrect.

6. The method of claim 1, further comprising selecting, by the computing system, a visual characteristic for the visual probe based on a visual characteristic of the fixation point presented on the display.

7. The method of claim 1, further comprising determining, by the computing system, to provide the session to the user in accordance with a session schedule, wherein the session schedule identifies a frequency over a time period at which the user is to be provided with the session.

8. The method of claim 1, wherein identifying the first visual stimulus and the second visual stimulus further comprises selecting, from a plurality of stimulus types, a first stimulus type for the session based on a second stimulus type selected for a prior session.

9. The method of claim 8, wherein the plurality of stimulus types including a text stimulus type, a scenic image stimulus type, a facial expression image stimulus type, or a video stimulus type.

10. The method of claim 1, further comprising identifying, by the computing system, an eye gaze of the user as toward one of the first visual stimulus associated with the chronic pain or the second visual stimulus being neutral with respect to the chronic pain.

11. The method of claim 1, further comprising determining, by the computing system, that the response is correct, responsive to identifying an eye gaze of the user as towards the second visual stimulus being neutral with respect to the chronic pain; and
wherein providing the feedback indication for the user further comprises generating the feedback indication based on the determination that the response is correct.

12. The method of claim 1, further comprising determining, by the computing system, that the response is incorrect, responsive to identifying an eye gaze of the user as towards the first visual stimulus being associated with the chronic pain; and
wherein providing the feedback indication for the user further comprises generating the feedback indication based on the determination that the response is incorrect.

13. The method of claim 1, further comprising modifying, by the computing system, a session schedule identifying a frequency over a time period at which the user is to be provided with the session based on a rate of correct responses by the user.

14. The method of claim 1, wherein providing the feedback indication further comprises providing the feedback indication based on a time elapsed between the presentation and the interaction.

15. The method of claim 1, wherein the user is on a medication to address the chronic pain associated with a condition, at least in partial concurrence with the session, and
wherein the chronic pain associated with the condition causes the user to have attention bias towards stimuli associated with the chronic pain, wherein the condition includes at least one of: rheumatoid arthritis, irritable bowel syndrome, fibromyalgia, or diabetic neuropathy.

16. A system for providing sessions to address chronic pain in users, comprising a computing system to:
identify, for a session to address chronic pain in a user, (i) a first visual stimulus associated with the chronic pain and (ii) a second visual stimulus being neutral with respect to the chronic pain;
present, relative to a fixation point on a display, the first visual stimulus at a first position and the second visual stimulus at a second position during a first portion of the session;
remove, from presentation on the display, the first visual stimulus and the second visual stimulus subsequent to elapsing of the first portion;
present, a visual probe corresponding to one of the first position or the second position relative to the fixation point, to direct the user to interact with the visual probe during a second portion of the session;
determine a response by the user to presentation of the visual probe; and
provide by the computing system, a feedback indication for the user based on the response by the user.

17. The system of claim 16, further comprising the computing system to:
receive, for each visual stimulus of a plurality of visual stimuli, an indication of a value identifying a degree of association of the corresponding visual stimulus with the chronic pain for the user based on at least one of: (i) an interaction with a user interface or (ii) an eye gaze with respect to the corresponding visual stimulus displayed on the user interface; and
wherein identifying the first visual stimulus further comprises selecting the first visual stimulus from the plurality of visual stimuli based on a corresponding value for the visual stimulus satisfying a threshold.

18. The system of claim 16, wherein the computing system is further configured to exclude, from a plurality of visual stimuli, at least one visual stimulus for presentation to the user, responsive to a corresponding value of the at least one visual stimulus not satisfying the threshold.

19. The system of claim 16, wherein the computing system is further configured to:
determine that the response by the user is correct, responsive to the user interacting with the visual probe where the second visual stimulus being neutral with respect to the chronic pain was presented on the display; and
generate the feedback indication based on the determination that the response is correct.

20. The system of claim 16, wherein the computing system is further configured to:
determine that the response by the user is incorrect, responsive to the user interacting on the display outside a threshold distance away from the where the second visual stimulus being neutral with respect to the chronic pain was presented on the display; and
generate the feedback indication based on the determination that the response is incorrect.

21. The system of claim 16, wherein the computing system is further configured to select a visual characteristic for the visual probe based on a visual characteristic of the fixation point presented on the display.

22. The system of claim 16, the computing system is further configured to provide the session to the user in accordance with a session schedule, wherein the session schedule identifies a frequency over a time period at which the user is to be provided with the session

23. The system of claim 16, wherein the computing system is further configured to select, from a plurality of stimulus types, a first stimulus type for the session based on a second stimulus type selected for a prior session.

24. The system of claim 23, wherein the plurality of stimulus types including a text stimulus type, a scenic image stimulus type, a facial expression image stimulus type, or a video stimulus type.

25. The system of claim 16, the computing system is further configured to identify an eye gaze of the user as toward one of the first visual stimulus associated with the chronic pain or the second visual stimulus being neutral with respect to the chronic pain.

26. The system of claim 16, wherein the computing system is further configured to:
determine that the response is correct, responsive to identifying an eye gaze of the user as towards the second visual stimulus being neutral with respect to the chronic pain; and
generate the feedback indication based on the determination that the response is correct.

27. The system of claim 16, wherein the computing system is further configured to:
determine that the response is incorrect, responsive to identifying an eye gaze of the user as towards the first visual stimulus being associated with the chronic pain; and
wherein providing the feedback indication for the user further comprises generating the feedback indication based on the determination that the response is incorrect.

28. The system of claim 16, wherein the computing system is further configured to modify a session schedule identifying a frequency over a time period at which the user is to be provided with the session based on a rate of correct responses by the user.

29. The system of claim 16, wherein the computing system is further configured to provide the feedback indication based on a time elapsed between the presentation and the interaction.

30. The system of claim 16, wherein the user is on a medication to address the chronic pain associated with a condition, at least in partial concurrence with the session, and
wherein the chronic pain associated with the condition causes the user to have attention bias towards stimuli associated with the chronic pain, wherein the condition includes at least one of: rheumatoid arthritis, irritable bowel syndrome, fibromyalgia, or diabetic neuropathy.
